(19) United States
(12) Patent Application Publication
(10) Pub. No.: US 2008/0169453 A1
(43) Pub. Date: Jul. 17, 2008

(54) Near infrared ray absorbing material and near infrared ray absorbing filter

(75) Inventors: Keizo Kimura, Kanagawa (JP); Katsuyoshi Yamakawa, Kanagawa (JP); Osamu Uchida, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM CORPORATION, Minato-ku (JP)

(21) Appl. No.: 11/889,825

(22) Filed: Aug. 16, 2007

(30) Foreign Application Priority Data
Sep. 22, 2006 (JP) 2006-257096

(51) Int. Cl. F21V 9/04; C07D 209/56; C07D 215/06; C07D 471/04; C07D 455/04; C07D 277/62

(52) U.S. Cl. 252/587; 548/427; 546/181; 544/350; 546/94; 548/156

(57) ABSTRACT

According to the present invention, there is provided a near infrared ray absorbing material comprising at least a first compound having a spectral absorption maximum wavelength of 470 nm or less in a range of 270 to 1600 nm in solution, and a second compound represented by the following formula (II-1) or (II-2).

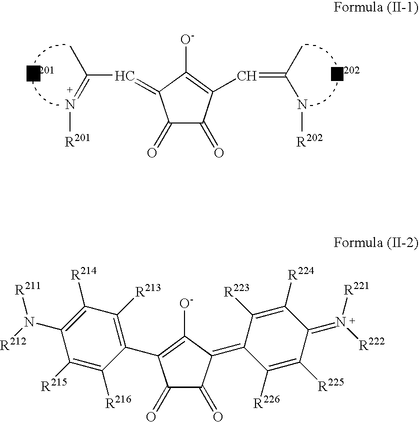

where in the formulas, R²⁰¹, R²⁰², R²¹¹, R²¹², R²²¹, and R²²² each represent, independently, a hydrogen atom, an aliphatic group, an aromatic group, or a heterocyclic group linked via a carbon atom; Z²⁰¹ and Z²⁰² represent a nonmetal atomic group necessary for forming a nitrogen-containing heterocycle; R²¹³ to R²¹⁶ and R²²³ to R²²⁶ represent a hydrogen atom or a substituent.

### CROSS-REFERENCE TO RELATED APPLICATION

This application claims priority under 35 USC 119 from Japanese Patent Application No. 2006-257096, the disclosure of which is incorporated by reference herein.

### BACKGROUND

1. Technical Field

The present invention relates to a near infrared ray absorbing material, more specifically, a near infrared ray absorbing material playing an important role for optoelectronics-related products such as a near infrared ray absorbing filter, a near infrared ray absorbing colored resin composition, a liquid crystal display element, an optical card, an optical recording medium, and a pair of safety glasses, and a near infrared ray absorbing filter comprising such a near infrared ray absorbing material.

2. Related Art

Near infrared ray absorbing dyes which do not substantially absorb visible light but absorb infrared rays are used in various optoelectronics products such as a near infrared ray absorbing filter. They are exposed to high temperature, high humidity, or photoirradiation conditions depending on the way of use, and the decomposition thereof becomes a problem in some cases.

As a technique for improving the resistance against these conditions by changing the structure of the dye, it was found that a naphthalocyanine dye having a specific structure (for example, refer to Japanese Patent Application Laid Open (JP-A) No. 2-4865, and U.S. Pat. Nos. 4,960,538 and 5,024,926) was satisfactory.

Moreover, techniques in which decomposition by light is suppressed by jointly using the near infrared ray absorbing dye and an ultraviolet ray absorbing material, have been known (for example, refer to JP-A No. 11-167350, JP-A No. 2001-133624, and JP-A No. 2005-181966).

### SUMMARY

The present invention has been made in view of the above circumstances and provides a near infrared ray absorbing material and a near infrared ray absorbing filter.

A first aspect of the present invention provides a near infrared ray absorbing material comprising at least a first compound having a spectral absorption maximum wavelength of 470 nm or less in a range of 270 to 1600 nm in solution, and a second compound represented by the following formula (II-1) or (II-2).

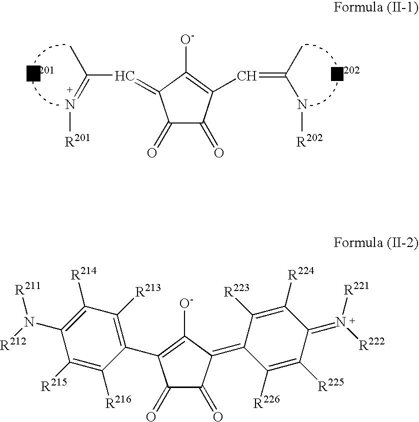

[In the formulas, R²⁰¹, R²⁰², R²¹¹, R²¹², R²²¹, and R²²² each represent, independently, a hydrogen atom, an aliphatic group, an aromatic group, or a heterocyclic group linked via a carbon atom. Z²⁰¹ and Z²⁰² represent a nonmetal atomic group necessary for forming a nitrogen-containing heterocycle. R²¹³ to R²¹⁶, and R²²³ to R²²⁶ represent a hydrogen atom or a substituent. R²¹³ and R²¹⁴, R²¹⁴ and R²¹¹, R²¹¹ and R²¹², R²¹² and R²¹⁵, R²¹⁵ and R²¹⁶, R²²³ and R²²⁴, R²²⁴ and R²²¹, R²²¹ and R²²², R²²² and R²²⁵, and R²²⁵ and R²²⁶ may be bonded with each other to form a ring.]

### DETAILED DESCRIPTION

Hereunder is a detailed description of embodiments of the present invention.

### <Near Infrared Ray Absorbing Material>

The near infrared ray absorbing material of the present invention comprises at least a first compound (hereunder, may be referred to as a compound (I)) having a spectral absorption maximum wavelength of 470 nm or less in a range of 270 to 1600 nm in solution, and a second compound (hereunder, may be referred to as a compound (II)) represented by the following formula (II-1) or (II-2). As a result, light resistance, near infrared ray absorbing power, and less discoloration after light deterioration can be all satisfied.

In the present invention, the aspect of “comprising at least” the compound (I) and the compound (II) is not specifically limited. Examples thereof include an aspect in which the compound (I) and the compound (II) are present in different layers, and an aspect in which the compound (I) and the compound (II) are present together in the same layer. Among these aspects, from the viewpoint of light resistance, the aspect in which the above compounds are present together in the same layer, is preferred.

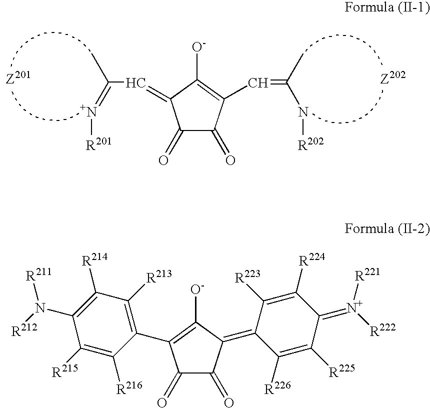

In the formulas, R²⁰¹, R²⁰², R²¹¹, R²¹², R²²¹, and R²²² each represent, independently, a hydrogen atom, an aliphatic group, an aromatic group, or a heterocyclic group linked via a carbon atom. Z²⁰¹ and Z²⁰² represent a nonmetal atomic group necessary for forming a nitrogen-containing heterocycle. R²¹³ to R²¹⁶, and R²²³ to R²²⁶ represent a hydrogen atom or a substituent. R²¹³ and R²¹⁴, R²¹⁴ and R²¹¹, R²¹¹ and R²¹², R²¹² and R²¹⁵, R²¹⁵ and R²¹⁶, R²²³ and R²²⁴, R²²⁴ and R²²¹, R²²¹ and R²²², R²²² and R²²⁵, and R²²⁵ and R²²⁶ may be bonded with each other to form a ring.

### (Spectral Absorption Maximum Wavelength)

Here is a description of the spectral absorption maximum wavelength.

The spectral absorption maximum wavelength is defined by the absorption spectrum in a solution. The solution may be formed from any solvent as long as the solvent can dissolve the compounds. The solvent may be an organic or inorganic solvent, or water, and may be used solely or as a mixture of a plurality of types thereof. In the present invention, as long as the solvent and the temperature satisfy conditions to dissolve the compounds, the spectral absorption maximum wavelength in any of such conditions may be any wavelength in a range defined by the present invention.

Examples of the organic solvent include an amide based solvent (such as N,N-dimethylformamide, N,N-dimethylacetamide, and 1-methyl-2-pyrrolidone), a sulfone based solvent (such as sulfolane), a sulfoxide based solvent (such as dimethyl sulfoxide), an ureido based solvent (such as tetramethylurea), an ether based solvent (such as dioxane, tetrahydrofuran, and cyclopentylmethylether), a ketone based solvent (such as acetone and cyclohexanone), a hydrocarbon based solvent (such as toluene, xylene, and n-decane), a halogen based solvent (such as tetrachloroethane, chlorobenzene, and chloronaphthalene), an alcohol based solvent (such as methanol, ethanol, isopropyl alcohol, ethylene glycol, cyclohexanol, and phenol), a pyridine based solvent (such as pyridine, γ-picoline, and 2,6-lutidine), an ester based solvent (such as ethyl acetate and butyl acetate), a carboxylic acid based solvent (such as acetic acid and propionic acid), a nitrile based solvent (such as acetonitrile), a sulfonic acid based solvent (such as methanesulfonic acid), and an amine based solvent (such as triethylamine and tributylamine).

Moreover, examples of the inorganic solvent include sulfuric acid and phosphoric acid.

Among these solvents, from the viewpoint of solubility, when the spectral absorption maximum wavelength of the compound (I) of the present invention is to be measured, the amide based solvent, the sulfone based solvent, the sulfoxide based solvent, the ureido based solvent, the ether based solvent, the ketone based solvent, the halogen based solvent, the alcohol based solvent, the ester based solvent, and the nitrile based solvent may be preferably used.

On the other hand, when the spectral absorption maximum wavelength of the compound (II) of the present invention is to be measured, the amide based solvent, the sulfone based solvent, the sulfoxide based solvent, the ureido based solvent, the ether based solvent, the hydrocarbon based solvent, the halogen based solvent, or the alcohol based solvent may be preferably used.

The concentration of the solution to measure the spectral absorption maximum wavelength may be any concentration as long as the maximum wavelength of spectral absorption can be ensured, and is preferably in a range of 1×10⁻¹³ to 1×10⁻⁷. The temperature is not specifically limited, but is preferably 0° C. to 80° C., and more preferably room temperature (25° C.) if there is no problem in the solubility of the compounds.

As the measuring equipment to measure the spectral absorption maximum wavelength, a normal absorption spectrometer (such as U-4100 spectrophotometer manufactured by Hitachi High-Technologies Corporation.) may be used.

### (Groups in the Present Invention)

Prior to the description of the compound, groups in the present invention are described in detail.

In the present description, the term “aliphatic group” means an alkyl group, a substituted alkyl group, an alkenyl group, a substituted alkenyl group, an alkynyl group, a substituted alkynyl group, an aralkyl group, and a substituted aralkyl group. The alkyl group may be branched, or may be in a ring form. The number of carbon atoms in the alkyl group is preferably 1 to 20, and more preferably 1 to 18. The alkyl portion in the substituted alkyl group is the same as the above alkyl group. The alkenyl group may be branched, or may be in a ring form. The number of carbon atoms in the alkenyl group is preferably 2 to 20, and more preferably 2 to 18. The alkenyl portion in the substituted alkenyl group is the same as the above alkenyl group. The alkynyl group may be branched, or may be in a ring form. The number of carbon atoms in the alkynyl group is preferably 2 to 20, and more preferably 2 to 18. The alkynyl portion in the substituted alkynyl group is the same as the above alkynyl group. The alkyl portion in the aralkyl group and the substituted aralkyl group is the same as the above alkyl group. The aryl portion in the aralkyl group and the substituted aralkyl group is the same as the following aryl group.

Examples of the substituent in the substituted alkyl group, the substituent in the substituted alkenyl group, the substituent in the substituted alkynyl group, or the substituent in the alkyl portion in the substituted aralkyl group include: halogen atoms (such as a chlorine atom, a bromine atom, and an iodine atom); alkyl groups [which refer to a linear, branched, or cyclic substituted or unsubstituted alkyl group, including an alkyl group (preferably, an alkyl group of 1 to 30 carbon atoms, such as methyl, ethyl, n-propyl, isopropyl, t-butyl, n-octyl, eicocyl, 2-chloroethyl, 2-cyanoethyl, and 2-ethylhexyl), a cycloalkyl group (preferably, a substituted or unsubstituted cycloalkyl group of 3 to 30 carbon atoms, such as cyclohexyl, cyclopentyl and 4-n-dodecylcyclohexyl), a bicycloalkyl group (preferably, a substituted or unsubstituted bicycloalkyl group of 5 to 30 carbon atoms, that is, a monovalent group in which one hydrogen atom is removed from bicycloalkane of 5 to 30 carbon atoms, such as bicyclo[1,2,2]heptane-2-yl and bicyclo[2,2,2]octane-3-yl), and furthermore, a tricyclo structure having a lot of cyclic structures. Alkyl groups in substituents (such as an alkyl group in an alkylthio group) described later, also refer to the alkyl group of the above concept.]; alkenyl groups [which refer to a linear, branched, or cyclic substituted or unsubstituted alkenyl group, including an alkenyl group (preferably, a substituted or unsubstituted alkenyl group of 2 to 30 carbon atoms such as vinyl, allyl, prenyl, geranyl, and oleyl), a cycloalkenyl group (preferably, a substituted or unsubstituted cycloalkenyl group of 3 to 30 carbon atoms, that is a monovalent group in which one hydrogen atom is removed from cycloalkene of 3 to 30 carbon atoms, such as 2-cyclopentene-1-yl and 2-cyclohexene-1-yl), a bicycloalkenyl group (a substituted or unsubstituted bicycloalkenyl group, preferably, a substituted or unsubstituted bicycloalkenyl group of 5 to 30 carbon atoms, that is, a monovalent group in which one hydrogen atom is removed from bicycloalkene having one double-bond, such as bicyclo[2,2,1]hept-2-en-1-yl and bicyclo[2,2,2]oct-2-en-4-yl)]; alkynyl groups (preferably, a substituted or unsubstituted alkynyl group of 2 to 30 carbon atoms, such as an ethynyl, propargyl, or trimethylsilylethynyl group);

aryl groups (preferably, a substituted or unsubstituted aryl group of 6 to 30 carbon atoms, such as phenyl, p-tolyl, naphthyl, m-chlorophenyl, and o-hexadecanoylaminophenyl); heterocyclic groups (preferably, a monovalent group in which one hydrogen atom is removed from a 5-membered or 6-membered substituted or unsubstituted aromatic or nonaromatic heterocyclic compound, and more preferably a 5-membered or 6-membered aromatic heterocyclic group of 3 to 30 carbon atoms, such as 2-furyl, 2-thienyl, 2-pyrimidinyl, and 2-benzothiazolyl); cyano groups; hydroxyl groups; nitro groups; carboxyl groups; alkoxy groups (preferably, a substituted or unsubstituted alkoxy group of 1 to 30 carbon atoms, such as methoxy, ethoxy, isopropoxy, t-butoxy, n-octyloxy, and 2-methoxy ethoxy); aryloxy groups (preferably, a substituted or unsubstituted aryloxy group of 6 to 30 carbon atoms such as phenoxy, 2-methylphenoxy, 4-t-butylphenoxy, 3-nitrophenoxy, and 2-tetradecanoylaminophenoxy); silyloxy groups (preferably, a silyloxy group of 3 to 20 carbon atoms, such as trimethylsilyloxy, and t-butyldimethylsilyloxy); heterocyclic oxy groups (preferably, a substituted or unsubstituted heterocyclic oxy group of 2 to 30 carbon atoms, such as 1-phenyltetrazole-5-oxy and 2-tetrahydropyranyloxy); acyloxy groups (preferably, a formyloxy group, a substituted or unsubstituted alkylcarbonyloxy group of 2 to 30 carbon atoms, and a substituted or unsubstituted arylcarbonyloxy group of 6 to 30 carbon atoms, such as formyloxy, acetyloxy, pivaloyloxy, stearoyloxy, benzoyloxy, and p-methoxy phenylcarbonyloxy); carbamoyloxy groups (preferably, a substituted or unsubstituted carbamoyloxy group of 1 to 30 carbon atoms, such as N,N-dimethylcarbamoyloxy, N,N-diethylcarbamoyloxy, morpholinocarbonyloxy, N,N-di-n-octylaminocarbonyloxy, and N-n-octylcarbamoyloxy); alkoxycarbonyloxy groups (preferably, a substituted or unsubstituted alkoxycarbonyloxy group of 2 to 30 carbon atoms, such as methoxy carbonyloxy, ethoxycarbonyloxy, t-butoxycarbonyloxy, and n-octylcarbonyloxy); aryloxycarbonyloxy groups (preferably, a substituted or unsubstituted aryloxycarbonyloxy group of 7 to 30 carbon atoms, such as phenoxycarbonyloxy, p-methoxy phenoxycarbonyloxy, and p-n-hexadecyloxyphenoxycarbonyloxy); amino groups (preferably, an amino group, a substituted or unsubstituted alkylamino group of 1 to 30 carbon atoms, and a substituted or unsubstituted anilino group of 6 to 30 carbon atoms, such as amino, methylamino, dimethylamino, anilino, N-methyl-anilino, and diphenylamino); acylamino groups (preferably, a formylamino group, a substituted or unsubstituted alkylcarbonylamino group of 1 to 30 carbon atoms, and a substituted or unsubstituted arylcarbonylamino group of 6 to 30 carbon atoms, such as formylamino, acetylamino, pivaloylamino, lauroylamino, benzoylamino, 3,4,5-tri-n-octyloxyphenylcarbonylamino); aminocarbonylamino groups (preferably, a substituted or unsubstituted aminocarbonylamino of 1 to 30 carbon atoms, such as carbamoylamino, N,N-dimethylaminocarbonylamino, N,N-diethylaminocarbonylamino, and morpholinocarbonylamino); alkoxycarbonylamino groups (preferably, a substituted or unsubstituted alkoxycarbonylamino group of 2 to 30 carbon atoms, such as methoxy carbonylamino, ethoxycarbonylamino, t-butoxycarbonylamino, n-octadecyloxycarbonylamino, and N-methyl-methoxy carbonylamino); aryloxycarbonylamino groups (preferably, a substituted or unsubstituted aryloxycarbonylamino group of 7 to 30 carbon atoms, such as phenoxycarbonylamino, p-chlorophenoxycarbonylamino, and m-n-octyloxyphenoxycarbonylamino); sulfamoylamino groups (preferably, a substituted or unsubstituted sulfamoylamino group of 0 to 30 carbon atoms, such as sulfamoylamino, N,N-dimethylaminosulfonylamino, and N-n-octylaminosulfonylamino); alkyl- and arylsulfonylamino groups (preferably, a substituted or unsubstituted alkylsulfonylamino of 1 to 30 carbon atoms and a substituted or unsubstituted arylsulfonylamino of 6 to 30 carbon atoms such as methylsulfonylamino, butylsulfonylamino, phenylsulfonylamino, 2,3,5-trichlorophenylsulfonylamino, and p-methylphenylsulfonylamino); mercapto groups;

alkylthio groups (preferably, a substituted or unsubstituted alkylthio group of 1 to 30 carbon atoms, such as methylthio, ethylthio, and n-hexadecylthio); arylthio groups (preferably, a substituted or unsubstituted arylthio of 6 to 30 carbon atoms, such as phenylthio, p-chlorophenylthio, and m-methoxy phenylthio); heterocyclic thio groups (preferably, a substituted or unsubstituted heterocyclic thio group of 2 to 30 carbon atoms, such as 2-benzothiazolylthio and 1-phenyltetrazole-5-ylthio); sulfamoyl groups (preferably, a substituted or unsubstituted sulfamoyl group of 0 to 30 carbon atoms, such as N-ethylsulfamoyl, N-(3-dodecyloxypropyl)sulfamoyl, N,N-dimethylsulfamoyl, N-acetylsulfamoyl, N-benzoylsulfamoyl, and N—(N′-phenylcarbamoyl)sulfamoyl); sulfo groups; alkyl- and arylsulfinyl groups (preferably, a substituted or unsubstituted alkylsulfinyl group of 1 to 30 carbon atoms and a substituted or unsubstituted arylsulfinyl group of 6 to 30 carbon atoms, such as methylsulfinyl, ethylsulfinyl, phenylsulfinyl, and p-methylphenylsulfinyl);

alkyl- or arylsulfonyl groups (preferably, a substituted or unsubstituted alkylsulfonyl group of 1 to 30 carbon atoms and a substituted or unsubstituted arylsulfonyl group of 6 to 30 carbon atoms, such as methylsulfonyl, ethylsulfonyl, phenylsulfonyl, and p-methylphenylsulfonyl); acyl groups (preferably, a formyl group, a substituted or unsubstituted alkylcarbonyl group of 2 to 30 carbon atoms, a substituted or unsubstituted arylcarbonyl group of 7 to 30 carbon atoms, or a substituted or unsubstituted heterocyclic carbonyl group of 4 to 30 carbon atoms linked with a carbonyl group via a carbon atom, such as acetyl, pivaloyl, 2-chloroacetyl, stearoyl, benzoyl, p-n-octyloxyphenylcarbonyl, 2-pyridylcarbonyl, and 2-furylcarbonyl); aryloxycarbonyl groups (preferably, a substituted or unsubstituted aryloxycarbonyl group of 7 to 30 carbon atoms, such as phenoxycarbonyl, o-chlorophenoxycarbonyl, m-nitrophenoxycarbonyl, and p-t-butylphenoxycarbonyl); alkoxycarbonyl groups (preferably, a substituted or unsubstituted alkoxycarbonyl group of 2 to 30 carbon atoms, such as methoxycarbonyl, ethoxycarbonyl, t-butoxycarbonyl, and n-octadecyloxycarbonyl); carbamoyl groups (preferably, a substituted or unsubstituted carbamoyl group of 1 to 30 carbon atoms, such as carbamoyl, N-methylcarbamoyl, N,N-dimethylcarbamoyl, N,N-di-n-octylcarbamoyl, and N-(methylsulfonyl)carbamoyl);

aryl or heterocyclic azo groups (preferably, a substituted or unsubstituted arylazo group of 6 to 30 carbon atoms and a substituted or unsubstituted heterocyclic azo group of 3 to 30 carbon atoms, such as phenylazo, p-chlorophenylazo, and 5-ethylthio-1,3,4-thiadiazol-2-ylazo); imide groups (preferably, N-succinimide and N-phthalimide); phosphino groups (preferably, a substituted or unsubstituted phosphino group of 2 to 30 carbon atoms such as dimethylphosphino, diphenylphosphino, and methylphenoxyphosphino); phosphinyl groups (preferably, a substituted or unsubstituted phosphinyl group of 2 to 30 carbon atoms, such as phosphinyl, dioctyloxyphosphinyl, and diethoxyphosphinyl); phosphinyloxy groups (preferably, a substituted or unsubstituted phosphinyloxy group of 2 to 30 carbon atoms, such as diphenoxyphosphinyloxy and dioctyloxyphosphinyloxy); phosphinylamino groups (preferably, a substituted or unsubstituted phosphinylamino group of 2 to 30 carbon atoms, such as dimethoxy phosphinylamino and dimethylaminophosphinylamino); silyl groups (preferably, a substituted or unsubstituted silyl group of 3 to 30 carbon atoms such as trimethylsilyl, t-butyldimethylsilyl, and phenyldimethylsilyl).

A hydrogen atom, if any, may be removed in the above functional group, and further substituted with the above group. Examples of such a functional group include an alkylcarbonylaminosulfonyl group, an arylcarbonylaminosulfonyl group, an alkylsulfonylaminocarbonyl group, and an arylsulfonylaminocarbonyl group. Examples thereof include methylsulfonylaminocarbonyl, p-methylphenylsulfonylaminocarbonyl, acetylaminosulfonyl, and benzoylaminosulfonyl group.

Examples of the substituent in the aryl portion in the substituted aralkyl group are the same as the following examples of the substituent in the substituted aryl group.

In the present specification, the term “aromatic group” means an aryl group and a substituted aryl group. Moreover, these aromatic groups may be condensed with an aliphatic ring, another aromatic ring, or a heterocycle. The number of carbon atoms in the aromatic group is preferably 6 to 40, more preferably 6 to 30, and yet more preferably 6 to 20. Moreover, among them, the aryl group is preferably phenyl or naphthyl, and particularly preferably phenyl.

The aryl portion in the substituted aryl group is the same as the above aryl group. Examples of the substituent in the substituted aryl group include those described above as the examples of “the substituent in the substituted alkyl group, the substituent in the substituted alkenyl group, the substituent in the substituted alkynyl group, or the substituent in the alkyl portion in the substituted aralkyl group”.

In the present invention, the heterocyclic group preferably contains a 5-membered or 6-membered saturated or unsaturated heterocycle. The heterocycle may be condensed with an aliphatic ring, an aromatic ring, or another heterocycle. Examples of the hetero atom in the heterocycle include B, N, O, S, Se, and Te. Among them, as the hetero atom in the heterocycle, N, O, and S are preferred. Preferably, the heterocycle has a free valent (monovalent) carbon atom (the heterocyclic group is linked via a carbon atom). The number of carbon atoms in the heterocyclic group is preferably 1 to 40, more preferably 1 to 30, and yet more preferably 1 to 20. Examples of the saturated heterocycle include a pyrrolidine ring, a morpholine ring, a 2-bora-1,3-dioxolane ring, and a 1,3-thiazolidine ring. Examples of the unsaturated heterocycle include an imidazole ring, a thiazole ring, a benzothiazole ring, a benzoxazole ring, a benzotriazole ring, a benzoselenazole ring, a pyridine ring, a pyrimidine ring, and a quinoline ring. The heterocyclic group may have a substituent. Examples of the substituent include those described above as the examples of “the substituent in the substituted alkyl group, the substituent in the substituted alkenyl group, the substituent in the substituted alkynyl group, or the substituent in the alkyl portion in the substituted aralkyl group”.

### (First Compound Having a Spectral Absorption Maximum Wavelength of 470 Nm or Less in a Range of 270 to 1600 nm in Solution)

The near infrared ray absorbing material of the present invention comprises at least a first compound (compound (I) having a spectral absorption maximum wavelength of 470 nm or less in a range of 270 to 1600 nm in solution.

From the viewpoint of spectral absorption property of the compound, the spectral absorption maximum wavelength in solution is preferably 430 nm or less, more preferably 410 nm or less, and yet more preferably 380 nm or less.

Examples of the compound (I) include benzotriazole compounds, benzophenone compounds, cinnamic acid compounds, thiazolidone compounds (such as compounds described in Japanese Patent Application publication (JP-B) No. 44-29627), 1,3-butadiene compounds (such as compounds described in JP-A No. 51-56620), salicylate ester compounds, and oxalyl dianilide compounds.

Among them, benzotriazole compounds, benzophenone compounds, cinnamic acid compounds, salicylate ester compounds, and oxalyl dianilide compounds are preferred, benzotriazole compounds, benzophenone compounds, cinnamic acid compounds, and salicylate ester compounds are more preferred, benzotriazole compounds, benzophenone compounds, and salicylate ester compounds are yet more preferred, benzotriazole compounds and benzophenone compounds are even yet more preferred, and benzotriazole compounds are the most preferred.

In the present invention, the compound (I) is preferably represented by any one of the following formulas (I-1) to (I-5).

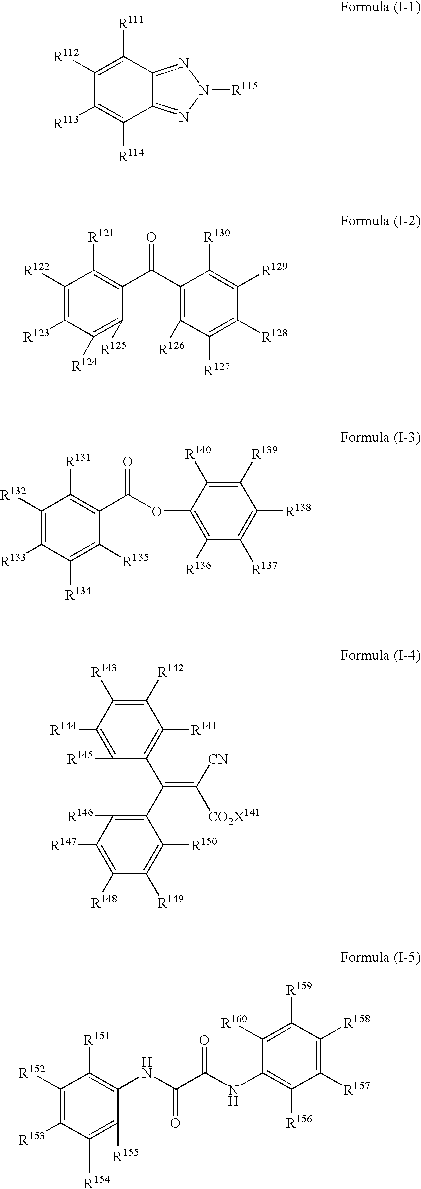

In the formulas, R¹¹¹ to R¹¹⁴, R¹²¹ to R¹³⁰, R¹³¹ to R¹⁴⁰, R¹⁴¹ to R¹⁵⁰, and R¹⁵¹ to R¹⁶⁰ each represent, independently, a hydrogen atom or a substituent, R¹¹⁵ represents a hydrogen atom, an aliphatic group, an aromatic group, or a heterocyclic group linked via a carbon atom, and X¹⁴¹ represents a hydrogen atom, an aliphatic group, an aromatic group, or a heterocyclic group linked via a carbon atom.

Here, adjacent groups among R¹¹¹ to R¹¹⁴, R¹²¹ to R¹³⁰, R¹³¹ to R¹⁴⁰, R¹⁴¹ to R¹⁵⁰, and R¹⁵¹ to R¹⁶⁰ substituted in a benzene ring in respective formulas, may be bonded with each other to form a ring.

Examples of the substituent represented by R¹¹¹ to R¹¹⁴, R¹²¹ to R¹³⁰, R¹³¹ to R¹⁴⁰, R¹⁴¹ to R¹⁵⁰, and R¹⁵¹ to R¹⁶⁰ include those described above as the examples of “the substituent in the substituted alkyl group, the substituent in the substituted alkenyl group, the substituent in the substituted alkynyl group, or the substituent in the alkyl portion in the substituted aralkyl group”.

R¹¹¹ to R¹¹⁴, R¹²¹ to R¹³⁰, R¹³¹ to R¹⁴⁰, R¹⁴¹ to R¹⁵⁰, and R¹⁵¹ to R¹⁶⁰ preferably represent a hydrogen atom, a halogen atom, an alkyl group, an alkenyl group, an alkynyl group, an aryl group, a cyano group, a hydroxyl group, a carboxyl group, an alkoxy group, an aryloxy group, a silyloxy group, an acyloxy group, a carbamoyloxy group, an alkoxycarbonyloxy group, an aryloxycarbonyloxy group, an amino group, an acylamino group, an aminocarbonylamino group, an alkoxycarbonylamino group, an aryloxycarbonylamino group, a sulfamoylamino group, an alkyl- and arylsulfonylamino group, a mercapto group, an alkylthio group, an arylthio group, a sulfamoyl group, a sulfo group, an alkyl- or arylsulfinyl group, an alkyl- or arylsulfonyl group, an acyl group, an aryloxycarbonyl group, an alkoxycarbonyl group, a carbamoyl group, an imide group, a phosphino group, a phosphinyl group, a phosphinyloxy group, a phosphinylamino group, or a silyl group.

R¹¹¹ to R¹¹⁴ more preferably represent, a hydrogen atom, a halogen atom, an alkyl group, an alkenyl group, an aryl group, a cyano group, a hydroxyl group, a carboxyl group, an alkoxy group, an aryloxy group, a silyloxy group, an amino group, an alkylthio group, an arylthio group, an imide group, or a silyl group, yet more preferably, a hydrogen atom, a halogen atom, an alkyl group, an aryl group, an alkoxy group, an aryloxy group, a silyloxy group, or an amino group, even yet more preferably, a hydrogen atom, a halogen atom, or an alkyl group, and most preferably, a hydrogen atom or a halogen atom.

R¹²¹ to R¹³⁰ more preferably represent a hydrogen atom, a halogen atom, an alkyl group, an alkenyl group, an aryl group, a cyano group, a hydroxyl group, a carboxyl group, an alkoxy group, an aryloxy group, a silyloxy group, an acyloxy group, an acylamino group, an aminocarbonylamino group, an alkoxycarbonylamino group, an aryloxycarbonylamino group, a sulfamoylamino group, an alkyl- or arylsulfonylamino group, a mercapto group, an alkylthio group, an arylthio group, a sulfamoyl group, a sulfo group, an alkyl- or arylsulfonyl group, an acyl group, an aryloxycarbonyl group, an alkoxycarbonyl group, a carbamoyl group, an imide group, or a silyl group, yet more preferably, a hydrogen atom, a halogen atom, an alkyl group, an aryl group, a hydroxyl group, an alkoxy group, an aryloxy group, an acyloxy group, an acylamino group, an alkyl- or arylsulfonylamino group, a sulfamoyl group, an acyl group, an aryloxycarbonyl group, an alkoxycarbonyl group, or a carbamoyl group, even yet more preferably, a hydrogen atom, a halogen atom, an alkyl group, a hydroxyl group, an alkoxy group, an acyloxy group, an acylamino group, an acyl group, an alkoxycarbonyl group, or a carbamoyl group, and even yet more preferably, a hydrogen atom, a halogen atom, an alkyl group, a hydroxyl group, an alkoxy group, an acylamino group, or an alkoxycarbonyl group. Moreover, most preferably, R¹²¹ is a hydroxyl group.

R¹³¹ to R¹⁴⁰ more preferably represent a hydrogen atom, a halogen atom, an alkyl group, an alkenyl group, an aryl group, a cyano group, a hydroxyl group, a carboxyl group, an alkoxy group, an aryloxy group, a silyloxy group, an acyloxy group, an acylamino group, an aminocarbonylamino group, an alkoxycarbonylamino group, an aryloxycarbonylamino group, a sulfamoylamino group, an alkyl- or arylsulfonylamino group, a mercapto group, an alkylthio group, an arylthio group, a sulfamoyl group, a sulfo group, an alkyl- or arylsulfonyl group, an acyl group, an aryloxycarbonyl group, an alkoxycarbonyl group, a carbamoyl group, an imide group, or a silyl group, more preferably, a hydrogen atom, a halogen atom, an alkyl group, an aryl group, a hydroxyl group, an alkoxy group, an aryloxy group, an acyloxy group, an acylamino group, an alkyl- or arylsulfonylamino group, a sulfamoyl group, an acyl group, an aryloxycarbonyl group, an alkoxycarbonyl group, or a carbamoyl group, even yet more preferably, a hydrogen atom, a halogen atom, an alkyl group, a hydroxyl group, an alkoxy group, an acyloxy group, an acylamino group, an acyl group, an alkoxycarbonyl group, and a carbamoyl group, and even yet more preferably, a hydrogen atom, a halogen atom, an alkyl group, a hydroxyl group, an alkoxy group, an acylamino group, or an alkoxycarbonyl group. Moreover, most preferably, R¹³¹ is a hydroxyl group.

R¹⁴¹ to R¹⁵⁰ more preferably represent a hydrogen atom, a halogen atom, an alkyl group, an aryl group, a hydroxyl group, an alkoxy group, an aryloxy group, a silyloxy group, an acyloxy group, a carbamoyloxy group, an alkoxycarbonyloxy group, an aryloxycarbonyloxy group, an amino group, an acylamino group, an aminocarbonylamino group, an alkoxycarbonylamino group, an aryloxycarbonylamino group, a sulfamoylamino group, an alkyl- or arylsulfonylamino group, an alkylthio group, an arylthio group, a sulfamoyl group, an acyl group, an aryloxycarbonyl group, an alkoxycarbonyl group, a carbamoyl group, or a silyl group, yet more preferably, a hydrogen atom, a halogen atom, an alkyl group, an aryl group, an alkoxy group, an aryloxy group, an amino group, an acylamino group, an alkylthio group, or an arylthio group, yet more preferably, a hydrogen atom, a halogen atom, an alkyl group, an alkoxy group, an amino group, or an acylamino group, and most preferably, a hydrogen atom, an alkoxy group, or an amino group.

R¹⁵¹ to R¹⁶⁰ more preferably represent a hydrogen atom, a halogen atom, an alkyl group, an aryl group, a cyano group, a hydroxyl group, an alkoxy group, an aryloxy group, an acyloxy group, an amino group, an acylamino group, a mercapto group, an alkylthio group, an arylthio group, a sulfamoyl group, an alkyl- or arylsulfinyl group, an alkyl- or arylsulfonyl group, an acyl group, an aryloxycarbonyl group, an alkoxycarbonyl group, or a silyl group, and yet more preferably, a hydrogen atom, a halogen atom, an alkyl group, an aryl group, a hydroxyl group, an alkoxy group, an acyloxy group, an amino group, an acylamino group, an arylthio group, an acyl group, an aryloxycarbonyl group, or an alkoxycarbonyl group. Most preferably, R¹⁵¹ is a hydroxyl group.

R¹¹⁵ represents a hydrogen atom, an aliphatic group, an aromatic group, or a heterocyclic group linked via a carbon atom, preferably, a hydrogen atom, an alkyl group, an alkenyl group, an alkynyl group, or an aryl group, more preferably, a hydrogen atom, an alkyl group of 1 to 30 carbon atoms, an alkenyl group of 2 to 30 carbon atoms, an alkynyl group of 2 to 30 carbon atoms, or an aryl group of 6 to 30 carbon atoms, yet more preferably, a hydrogen atom, an alkyl group of 1 to 25 carbon atoms, an alkenyl group of 2 to 25 carbon atoms, or an aryl group of 6 to 25 carbon atoms, yet more preferably, an alkyl group of 1 to 22 carbon atoms or an aryl group of 6 to 22 carbon atoms, and yet more preferably, an aryl group of 6 to 20 carbon atoms, and most preferably, an orthohydroxyphenyl group of 6 to 20 carbon atoms.

X¹⁴¹ represents a hydrogen atom, an aliphatic group, an aromatic group, or a heterocyclic group linked via a carbon atom, preferably, a hydrogen atom, an alkyl group, an alkenyl group, an alkynyl group, or an aryl group, more preferably, a hydrogen atom, an alkyl group of 1 to 30 carbon atoms, an alkenyl group of 2 to 30 carbon atoms, an alkynyl group of 2 to 30 carbon atoms, or an aryl group of 6 to 30 carbon atoms, yet more preferably, a hydrogen atom, an alkyl group of 1 to 25 carbon atoms, an alkenyl group of 2 to 25 carbon atoms, or an aryl group of 6 to 25 carbon atoms, yet more preferably, an alkyl group of 1 to 22 carbon atoms or an aryl group of 6 to 22 carbon atoms, and most preferably, an alkyl group of 1 to 18 carbon atoms.

In the present invention, preferably, a plurality of types of the above first compounds are contained.

Moreover, in the present invention, preferably, at least one type of the first compounds is a compound represented by the above formula (I-1).

Furthermore, in the present invention, more preferably, at least one type of the first compounds is a compound represented by any one of the above formulas (I-2) to (I-5).

Furthermore, in the present invention, yet more preferably, if a plurality of types of the first compounds are contained, at least one type is a compound represented by the above formula (I-1), and the other compound is a compound represented by any one of the above formulas (I-2) to (I-5).

By having the above structure, the light resistance is more effectively improved.

Hereunder, as specific examples of the “compound having a spectral absorption maximum wavelength of 470 nm or less in a range of 270 to 1600 nm in solution”, exemplary compounds (I-1) to (I-97) are shown. However, the present invention is not limited to these.

### (Compounds Included in Formula (I-1))

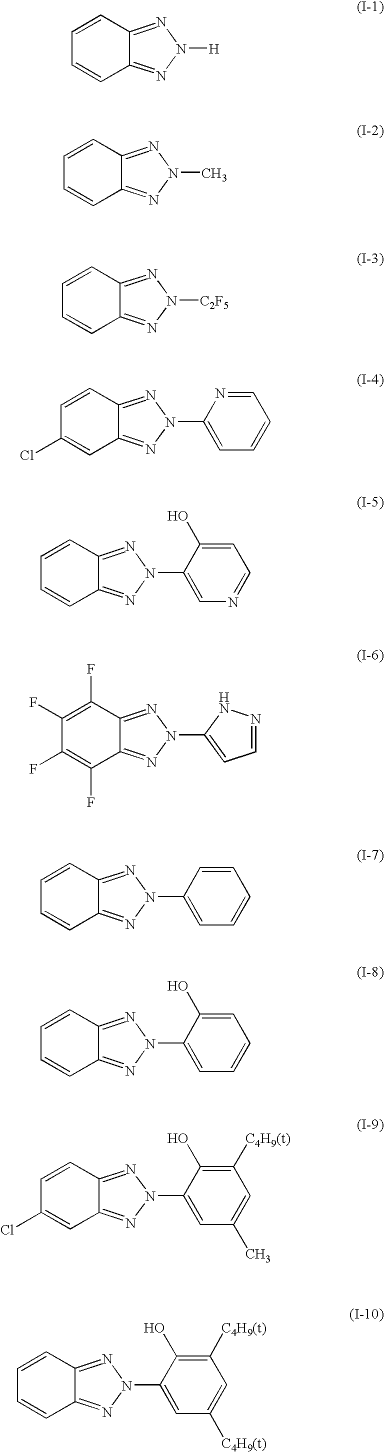

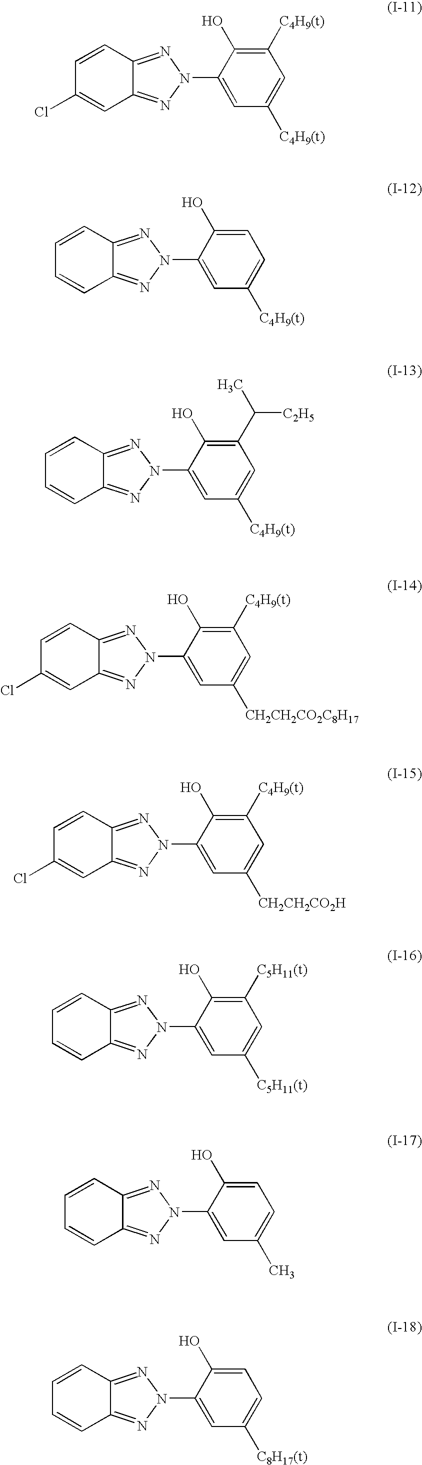

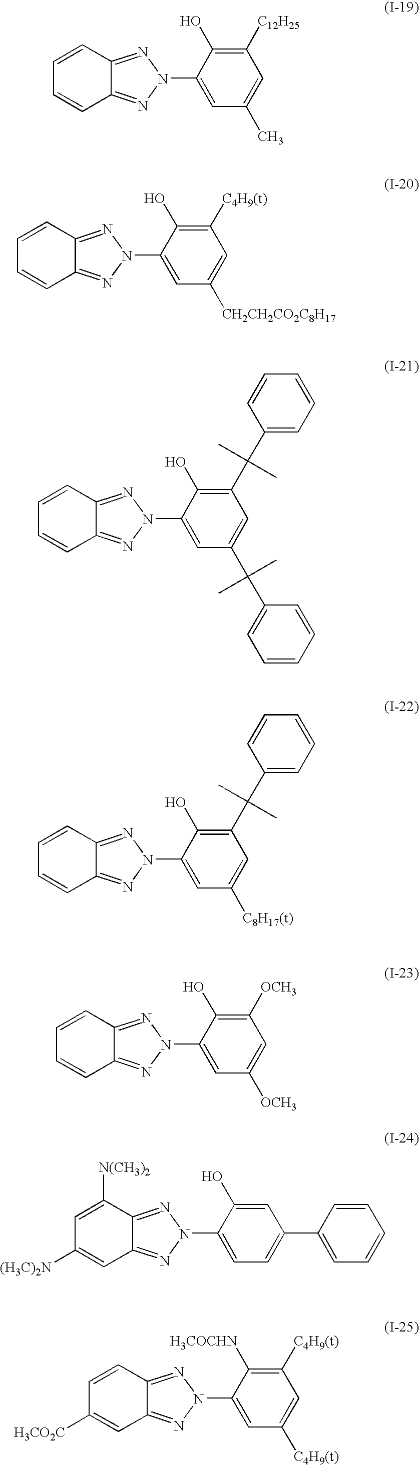

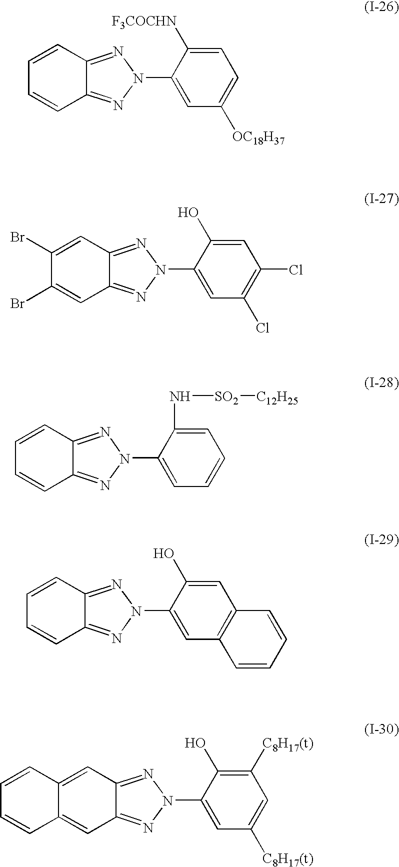

### (Compounds Included in Formula (I-2))

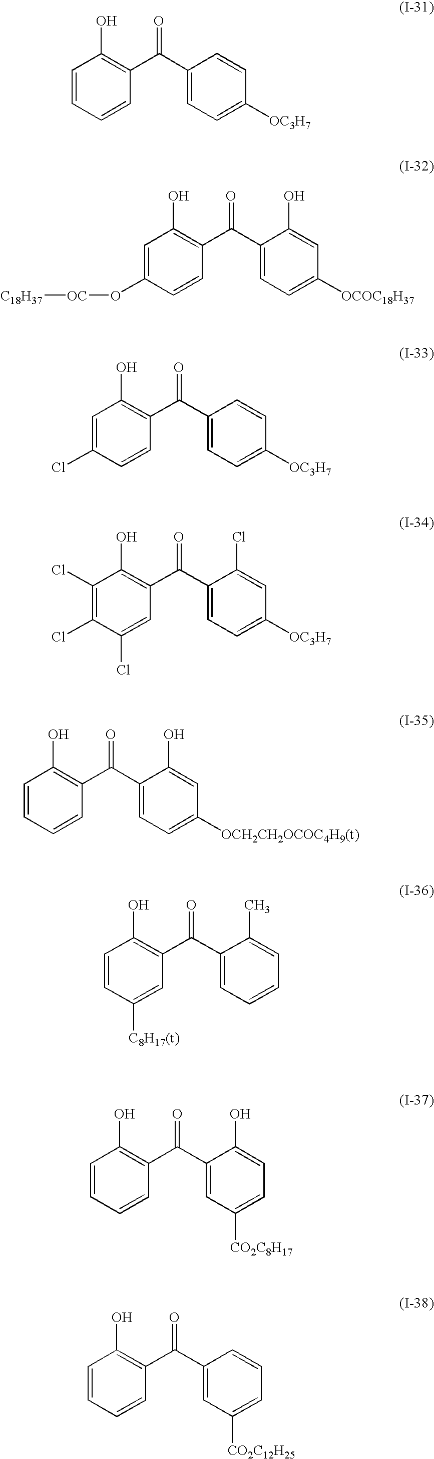

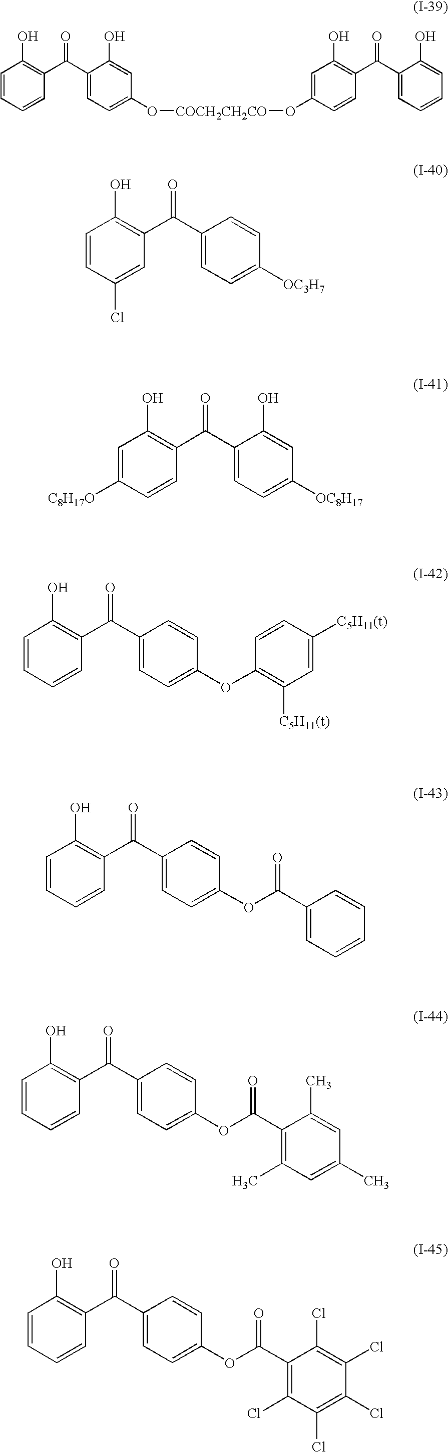

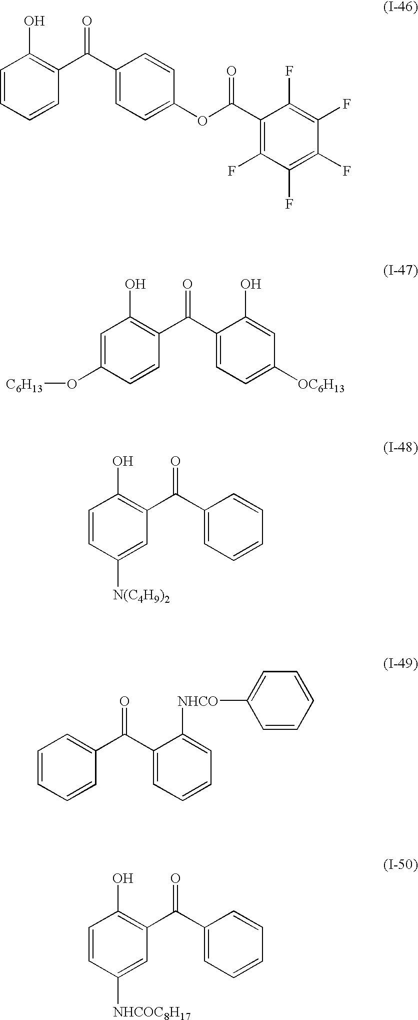

### (Compounds Included in Formula (I-3))

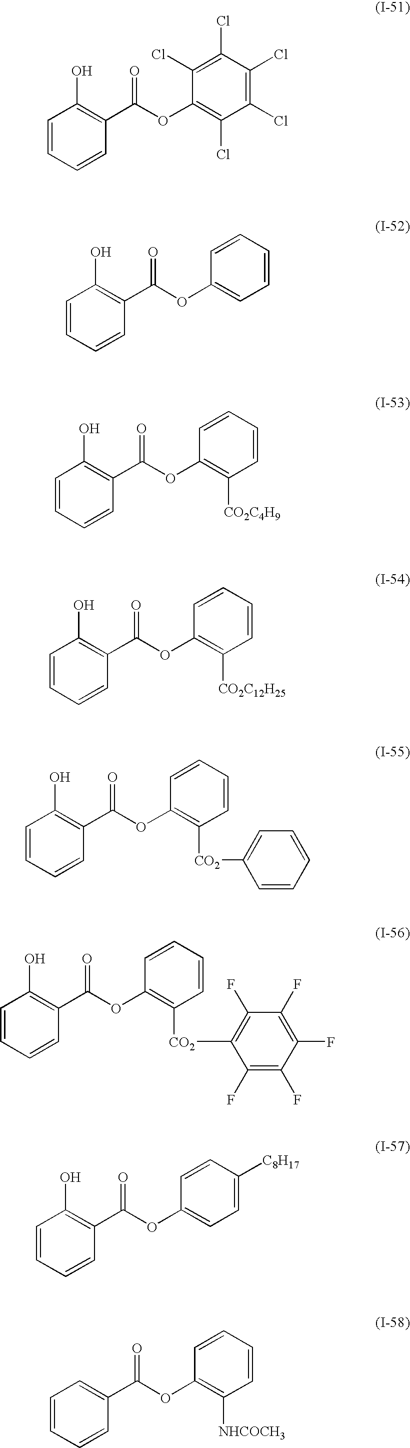

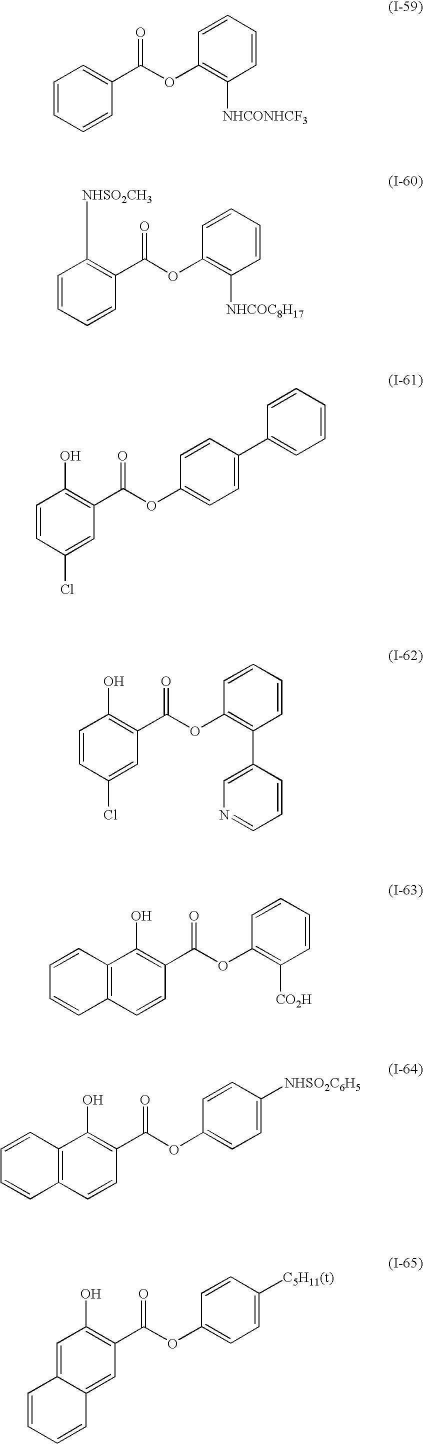

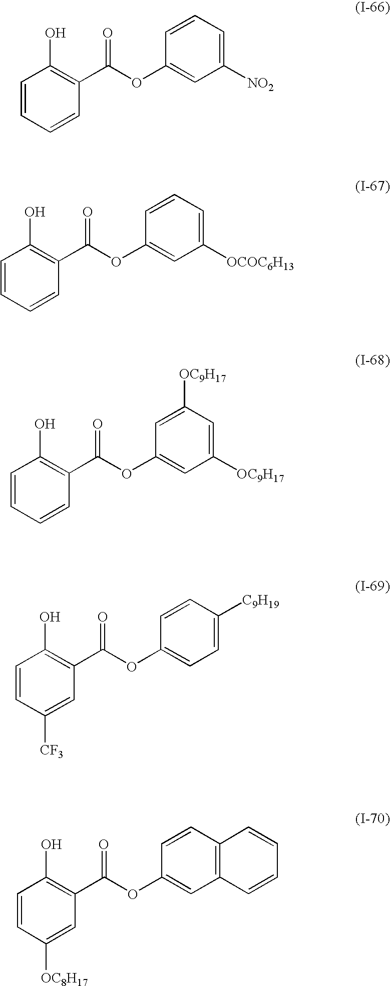

### (Compounds Included in Formula (I-4))

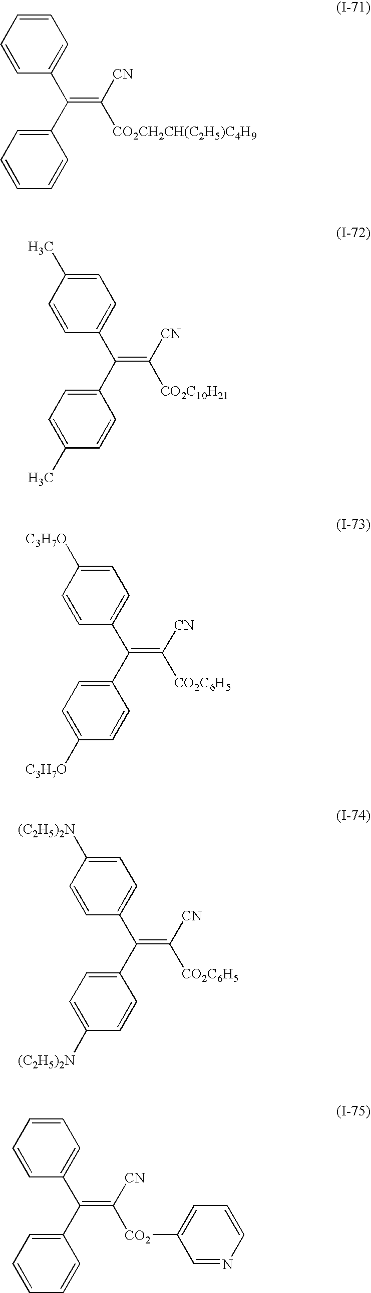

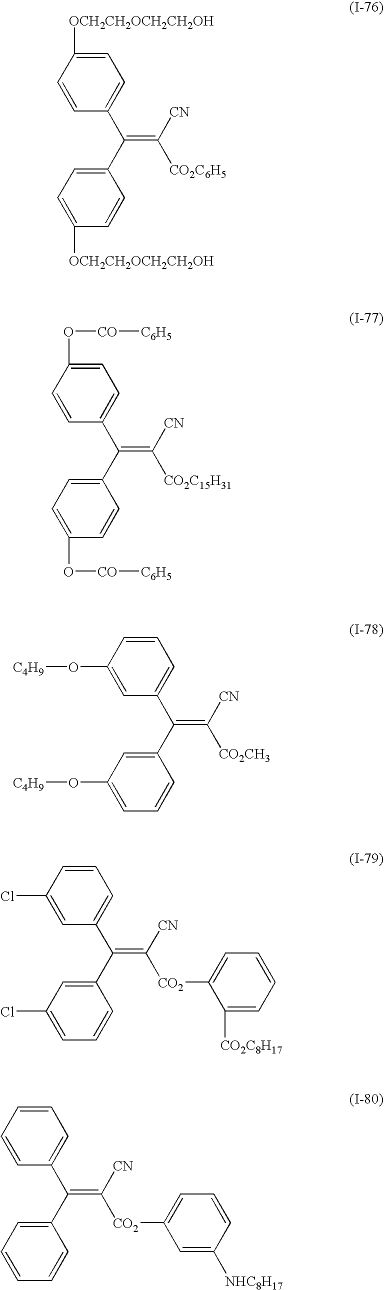

### (Compounds Included in Formula (I-5))

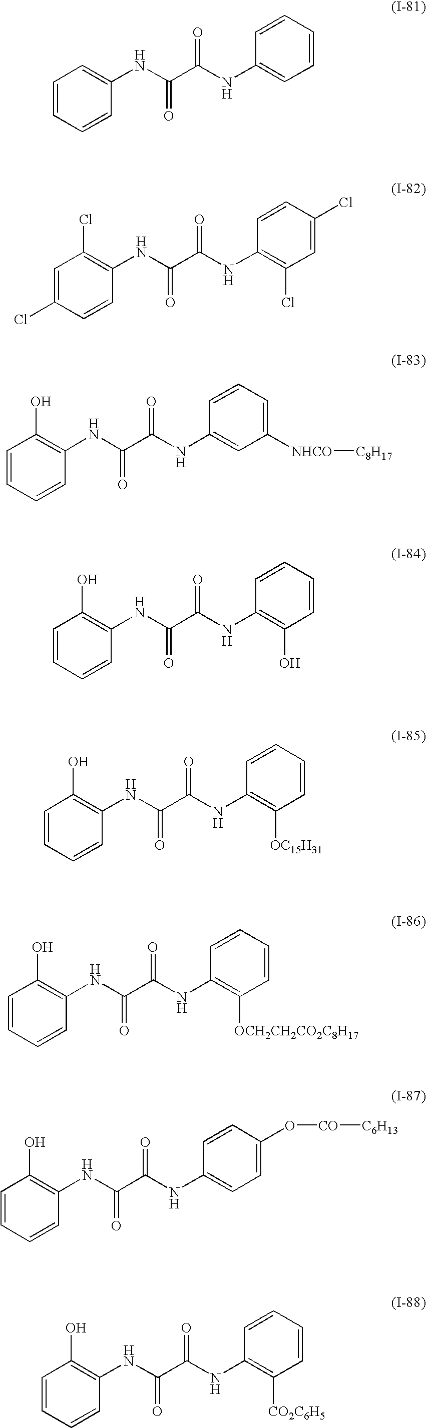

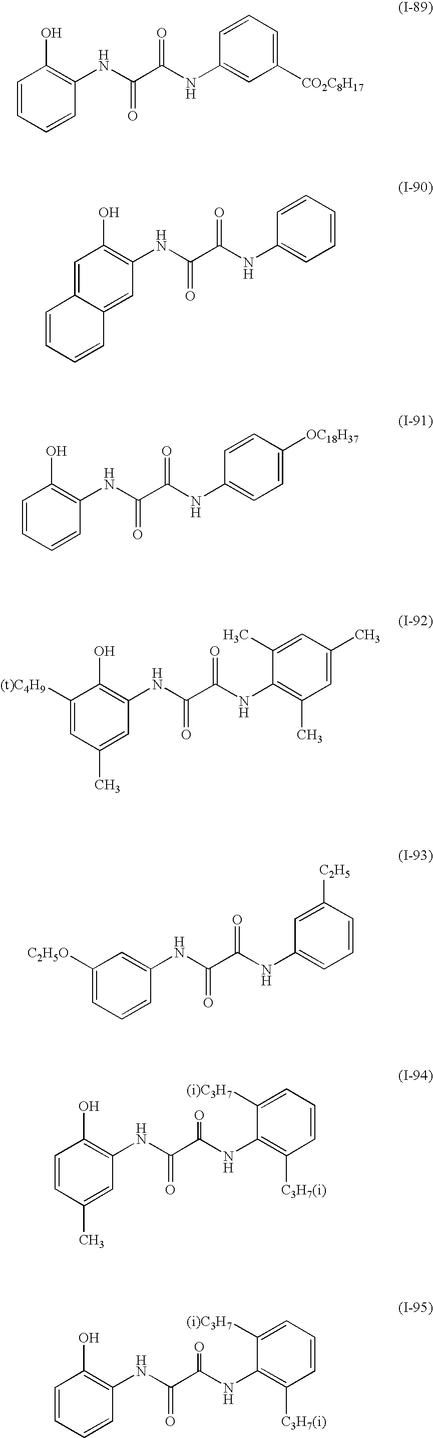

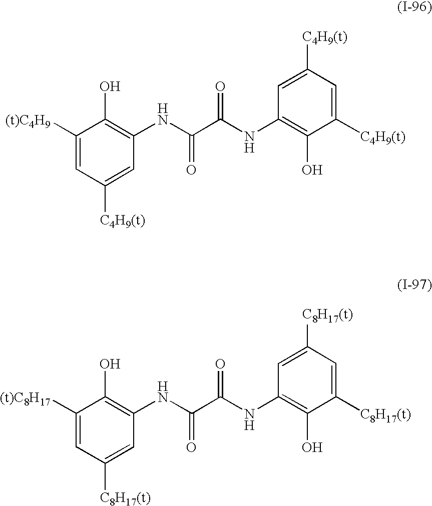

These compounds can be readily synthesized according to or based on methods described in JP-B No. 50-25337, U.S. Pat. No. 3,785,827, JP-A No. 5-4449, JP-B No. 48-30492, and The Journal of Organic Chemistry vol. 23, p 1344 (1958). Moreover, the compounds represented by the above formula (I-1) are commercially available, for example, under a product name of “TINUVIN 109” by Ciba Specialty Chemicals.

### <Compounds Represented by the Formula (II-1) or (II-2)>

The near infrared ray absorbing material of the present invention comprises at least a second compound represented by the following formula (II-1) or (II-2).

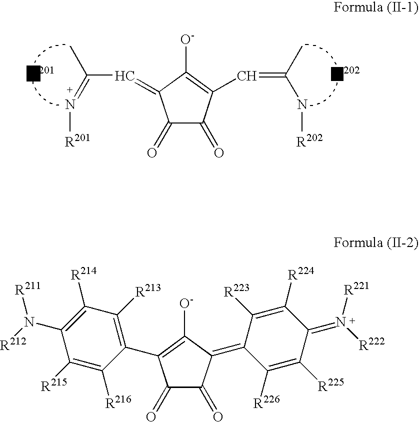

In the formulas, R²⁰¹, R²⁰², R²¹¹, R²¹², R²²¹, and R²²² each represent, independently, a hydrogen atom, an aliphatic group, an aromatic group, or a heterocyclic group linked via a carbon atom.

R²⁰¹ and R²⁰² preferably represent a hydrogen atom, an aliphatic group of 1 to 30 carbon atoms, an aromatic group of 6 to 30 carbon atoms, or a heterocyclic group of 2 to 30 carbon atoms linked via a carbon atom, more preferably, a hydrogen atom, an alkyl group or alkenyl group of 1 to 20 carbon atoms, a phenyl group or naphthyl group of 6 to 20 carbon atoms, or a 5-membered or 6-membered heterocyclic group of 2 to 20 carbon atoms linked via a carbon atom, yet more preferably, a hydrogen atom, an alkyl group of 1 to 20 carbon atoms or a phenyl group of 6 to 20 carbon atoms, and even yet more preferably, a hydrogen atom or an alkyl group of 1 to 20 carbon atoms.

R²¹¹, R²¹², R²²¹, and R²²² preferably represent an aliphatic group, an aromatic group, or a heterocyclic group linked via a carbon atom, more preferably, an alkyl group or alkenyl group of 1 to 30 carbon atoms, a phenyl group or naphthyl group of 6 to 30 carbon atoms, or a heterocyclic group of 2 to 30 carbon atoms linked via a carbon atom, yet more preferably, an alkyl group of 1 to 20 carbon atoms, a phenyl group of 6 to 20 carbon atoms, or a heterocyclic group of 2 to 20 carbon atoms linked via a carbon atom, even yet more preferably, an alkyl group of 1 to 15 carbon atoms or a phenyl group of 6 to 15 carbon atoms, and most preferably, an alkyl group of 1 to 10 carbon atoms.

Z²⁰¹ and Z²⁰² represent a nonmetal atomic group necessary for forming a nitrogen-containing heterocycle. The nitrogen-containing heterocycle formed by Z²⁰¹ and Z²⁰² is preferably a 5-membered or 6-membered nitrogen-containing heterocycle, more preferably, a quinoline ring, a benzothiazole ring, a naphthothiazole ring, a benzooxazole ring, an indolenine ring, or a benzoindolenine ring, and yet more preferably, a quinoline ring, a benzothiazole ring, a naphthothiazole ring, an indolenine ring, or a benzoindolenine ring

R²¹³ to R²¹⁶, and R²²³ to R²²⁶ represent a hydrogen atom or a substituent. R²¹³ and R²¹⁴, R²¹⁴ and R²¹¹, R²¹¹ and R²¹², R²¹² and R²¹⁵, R²¹⁵ and R²¹⁶, R²²³ and R²²⁴, R²²⁴ and R²²¹, R²²¹ and R²²², R²²² and R²²⁵, and R²²⁵ and R²²⁶ may be bonded with each other to form a ring.

Examples of the substituent include those described above as the examples of “the substituent in the substituted alkyl group, the substituent in the substituted alkenyl group, the substituent in the substituted alkynyl group, or the substituent in the alkyl portion in the substituted aralkyl group”. If R²¹³ to R²¹⁶ and R²²³ to R²²⁶ are the substituent, they preferably represent a halogen atom, an alkyl group, an alkenyl group, an alkynyl group, an aryl group, a heterocyclic group, a cyano group, a hydroxyl group, a nitro group, a carboxyl group, an alkoxy group, an aryloxy group, a silyloxy group, a heterocyclic oxy group, an acyloxy group, a carbamoyloxy group, an alkoxycarbonyloxy group, an aryloxycarbonyloxy group, an amino group, an acylamino group, an aminocarbonylamino group, an alkoxycarbonylamino group, an aryloxycarbonylamino group, a sulfamoylamino group, an alkyl- or arylsulfonylamino group, a mercapto group, an alkylthio group, an arylthio group, a heterocyclic thio group, a sulfamoyl group, a sulfo group, an alkyl- or arylsulfinyl group, an alkyl- or arylsulfonyl group, an acyl group, an aryloxycarbonyl group, an alkoxycarbonyl group, a carbamoyl group, an imide group, a phosphino group, a phosphinyl group, a phosphinyloxy group, a phosphinylamino group, or a silyl group,

more preferably, a halogen atom, an alkyl group, an aryl group, a cyano group, a hydroxyl group, a nitro group, a carboxyl group, an alkoxy group, an aryloxy group, a silyloxy group, an acyloxy group, a carbamoyloxy group, an amino group, an acylamino group, a mercapto group, an alkylthio group, an arylthio group, a sulfamoyl group, a sulfo group, an alkyl- or arylsulfinyl group, an alkyl- or arylsulfonyl group, an acyl group, an aryloxycarbonyl group, an alkoxycarbonyl group, a carbamoyl group, an imide group, a phosphino group, a phosphinyl group, a phosphinyloxy group, a phosphinylamino group, or a silyl group, yet more preferably, a halogen atom, an alkyl group, an aryl group, a cyano group, a hydroxyl group, a carboxyl group, an alkoxy group, an aryloxy group, a silyloxy group, an amino group, an acylamino group, an alkylthio group, an arylthio group, an acyl group, a carbamoyl group, or a silyl group,

yet more preferably, a halogen atom, a cyano group, a hydroxyl group, a carboxyl group, an alkyl group or alkoxy group of 1 to 20 carbon atoms, a silyloxy group, an amino group, an acylamino group, an alkylthio group, an acyl group, a carbamoyl group, a silyl group, an aryl group or aryloxy group of 6 to 20 carbon atoms, or an arylthio group, even yet more preferably, a halogen atom, a cyano group, a hydroxyl group, an alkyl group or alkoxy group of 1 to 12 carbon atoms, an amino group, an aryl group or aryloxy group of 6 to 12 carbon atoms, and yet more preferably, a halogen atom, a hydroxyl group, an alkyl group or alkoxy group of 1 to 8 carbon atoms.

Among compounds represented by the formula (II-1), compounds in which nitrogen-containing heterocycle formed by Z²⁰¹ and Z²⁰² is any one of a quinoline ring, a benzothiazole ring, a naphthothiazole ring, an indolenine ring, or a benzoindolenine ring, and R²⁰¹ and R²⁰² are a hydrogen atom or an alkyl group of 1 to 15 carbon atoms, are preferred. Compounds in which nitrogen-containing heterocycle formed by Z²⁰¹ and Z²⁰² is any one of a quinoline ring, a naphthothiazole ring, or a benzoindolenine ring, and R²⁰¹ and R²⁰² are a hydrogen atom or an alkyl group of 1 to 10 carbon atoms, are more preferred.

Among compounds represented by the formula (II-2), compounds in which R²¹³ to R²¹⁵ and R²²³ to R²²⁵ are a hydrogen atom, an alkyl group of 1 to 3 carbon atoms, or a halogen atom, R²¹⁶, and R²²⁶ are a hydroxyl group or an acylamino group, and R²¹¹, R²¹², R²²¹, and R²²² are an alkyl group of 1 to 10 carbon atoms, are preferred.

In the present invention, compounds represented by the above formula (II-2) are preferred among compounds represented by the formula (II-1) or (II-2).

Among compounds represented by the above formula (II-2), compounds in which R²¹³ and R²²³ are a hydrogen atom, R²¹⁴ and R²¹¹, R²¹⁵ and R²¹², R²²⁴ and R²²¹, and R²²⁵ and R²²² are alkyl groups which are bonded with each other to form a 5-membered or 6-membered ring, R²¹⁶ and R²²⁶ are a hydroxyl group, or alternatively, compounds in which R²¹¹, R²¹², R²²¹, and R²²² are an alkyl group, R²¹³ to R²¹⁵ and R²²³ to R²²⁵ are a hydrogen atom, and R²¹⁶ and

R²²⁶ are a hydroxyl group, are more preferred.

Hereunder, as specific examples of the compounds represented by the formula (II-1) or (II-2), exemplary compounds (II-1) to (II-30) are shown. However, the present invention is not limited to these.

### (Compounds Represented by Formula (II-1))

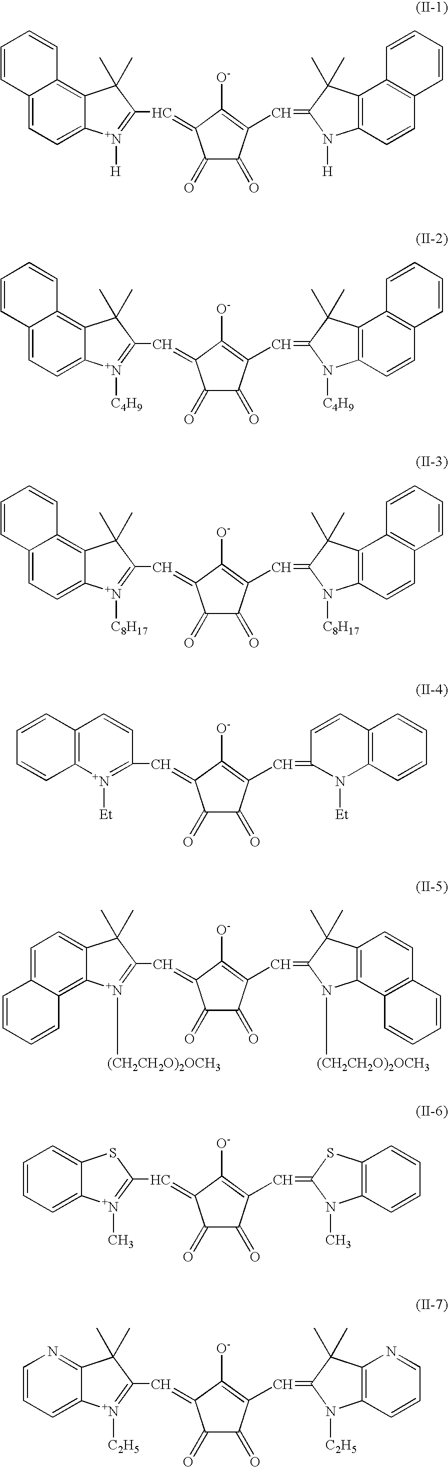

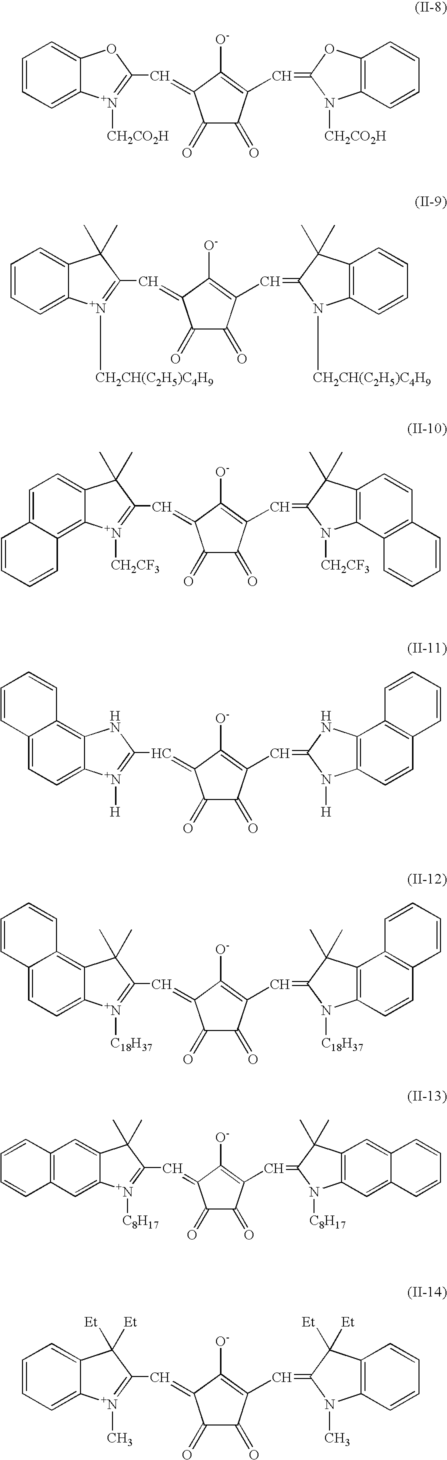

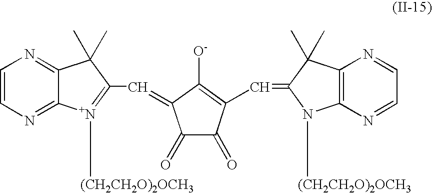

### (Compounds Represented by Formula (II-2))

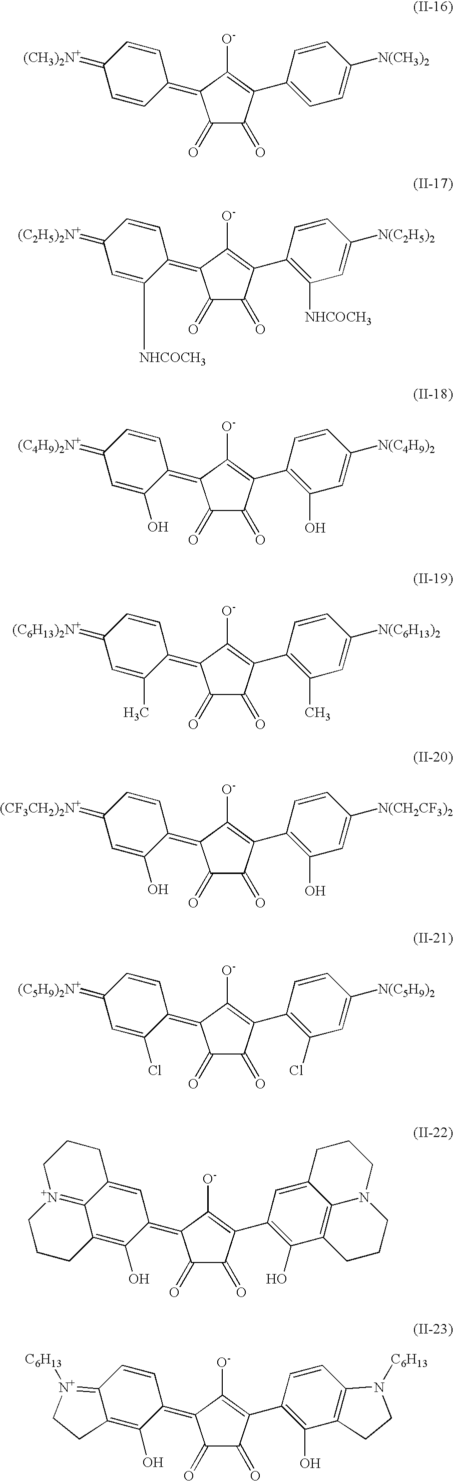

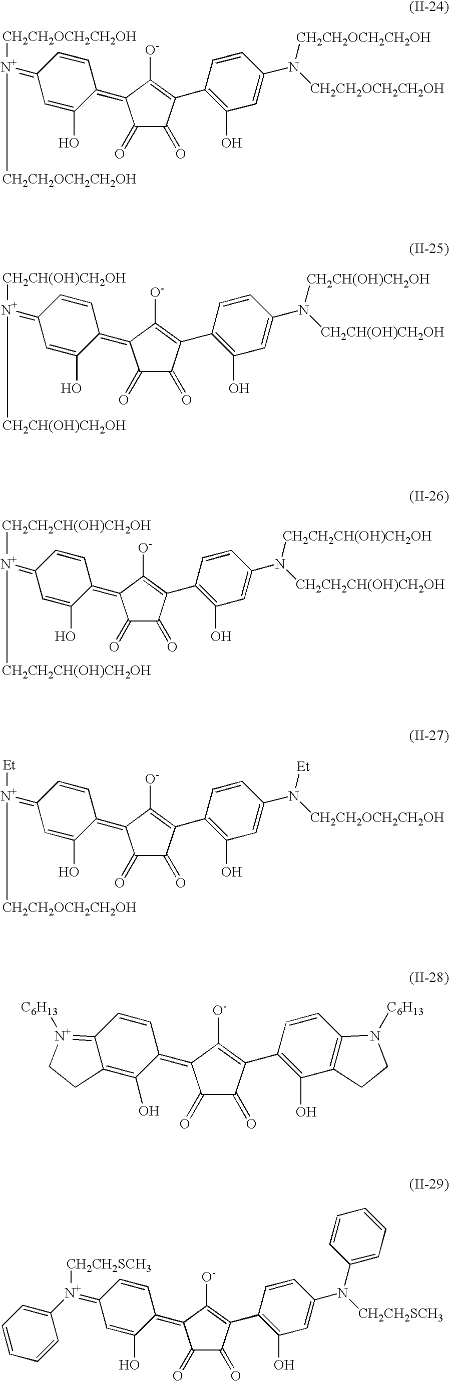

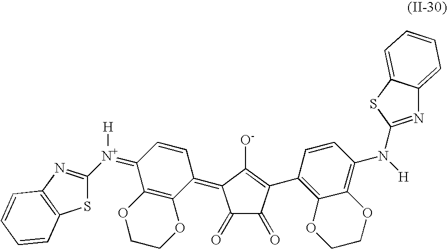

The compounds represented by the formula (II-1) or (II-2) may be synthesized with reference to methods described in, for example. JP-A No: 2-84383, JP-B No. 51-41061, JP-A No. 5-313305, and Liebigs Annalen der Chemie p 935-939 (1993).

For example, when a compound represented by the formula (II-2) is to be formed, the mole ratio of an aniline compound to be used with respect to croconic acid is preferably 1.5 to 3, more preferably 1.8 to 2.5, and yet more preferably, 1.9 to 2.1.

Examples of the reaction solvent include amide based solvents (such as N,N-dimethylformamide, N,N-dimethylacetamide, and 1-methyl-2-pyrrolidone), sulfone based solvents (such as sulfolane), sulfoxide based solvents (such as dimethyl sulfoxide), ureido based solvents (such as tetramethylurea), ether based solvents (such as dioxane and cyclopentylmethylether), ketone based solvents (such as acetone and cyclohexanone), hydrocarbon based solvents (such as toluene, xylene, mesitylene, and n-octane), halogen based solvents (such as tetrachloroethane and chlorobenzene), alcohol based solvents (such as 1-butanol, ethylene glycol, and cyclohexanol), and carboxylic acid based solvents (such as acetic acid), either solely or in combination.

As to the reaction solvent, an absence of solvent, hydrocarbon based solvents, halogen based solvents, alcohol based solvents, ether based solvents, and carboxylic acid based solvents are preferred, and hydrocarbon based solvents, halogen based solvents, and alcohol based solvents are more preferred.

The reaction temperature is within a range of 0 to 250° C., preferably 50 to 200° C., and more preferably 60 to 150° C. The reaction time is within a range of 5 minutes to 30 hours.

During the reaction, it is also preferred to remove water as a by-product out of the system. A method of removing by evaporation under a low or normal pressure either solely or together with a solvent, a method of using an absorbent such as a molecular sieve, a method of using a dehydrating and condensing agent such as acetic anhydride, and the like are preferably used.

The aniline compounds serving as a raw material may be synthesized with reference to a method described in, for example, JP-A No. 10-29976.

The total number of moles of the compound (compound (I)) having a spectral absorption maximum wavelength of 470 nm or less in a range of 270 to 1600 nm in solution contained in the near infrared ray absorbing material of the present invention is preferably 0.1 to 2.0 moles with respect to 1 mole of the second compound (compound (II)) represented by the formula (II-1) or (II-2), more preferably 0.1 to 1.0 moles, and yet more preferably 0.1 to 0.5 moles. By having the above range, light resistance is more effectively improved, and satisfying both light resistance and less discoloration after light deterioration is facilitated.

Moreover, the content of the compound (II) in the near infrared ray absorbing material of the present invention is not specifically limited, however, this is preferably 10⁻¹⁰ to 20 mass % in the total solid, more preferably 10⁻⁷ to 5 mass %, and yet more preferably 10⁻⁴ to 3 mass %. By having the above range of the content of compound (II), a satisfactory near infrared ray absorbing power can be obtained.

In the present invention, the following combinations of compound (I) and compound (II) are preferred aspects.

a) An aspect in which the compound (II) is a compound represented by the formula (II-2), and the compound (I) is a compound represented by the formula (I-1).

b) An aspect in which the compound (II) is a compound represented by the formula (II-2) and the compound (I) is a compound of two or more types represented by the formula (I-1).

c) An aspect in which the compound (II) is a compound represented by the formula (II-2), and the compound (I) is a combination of a compound represented by the formula (I-1) and a compound represented by any one of the above formulas (I-2) to (I-5).

### (Method of Producing Near Infrared Ray Absorbing Material)

The near infrared ray absorbing material of the present invention may be obtained by the following methods, for example.

(1) a method of dissolving or dispersing the compound (I) and the compound (II) in a solvent (such as chloroform, methylene chloride, toluene, acetone, methylethylketone, cyclohexanone, ethyl acetate, dibutylether, tetrahydrofuran, dimethylformamide, and water).

(2) a method of heating and kneading the compound (I) and the compound (II) with a resin (such as an ABS resin, a polyethylene resin, a polypropylene resin, a polyvinyl chloride resin, a polycarbonate resin, a polystyrene resin, a polyacrylonitrile resin, a methacrylonitrile resin, a polymethacrylate resin, and a polyester resin).

(3) a method in which the compound (I) and the compound (II) are dissolved or dispersed in the above solvent, then added with the above resin, followed by heating and dissolving, and then formed into a thin film or solidified as it is.

(4) a method of coating or hardcoating a solution having the compound (I) and the compound (II) dissolved or dispersed in the solvent, onto a paper, a resin sheet, a resin, a film, a glass, a metal plate, or the like.

(5) a method of polymerizing the compound (I) and the compound (II) as it is, as fine particles, a solution, or a mixture with a monomer.

In the above respective methods, a binder or another compound may be jointly used as required.

### (Method of Using Near Infrared Ray Absorbing Material)

The near infrared ray absorbing material of the present invention can be used for various applications. That is, it can be used for an optical recording medium for long wavelength lasers, a recording medium for invisible printing, an optical filter, a filter for construction and agriculture, a coating material, and the like. Among these, it can be preferably used for an optical filter, a filter for construction and agriculture, and a coating material, and more preferably for an optical filter.

Since the near infrared ray absorbing material of the present invention contains the compound (I) and the compound (II), light resistance, near infrared ray absorbing power, and less discoloration after light deterioration can be all satisfied, and it can also be used for new applications.

### <Near Infrared Ray Absorbing Filter>

The near infrared ray absorbing filter of the present invention comprises the near infrared ray absorbing material of the present invention. The near infrared ray absorbing filter of the present invention can be produced by the method described in the above method of producing near infrared ray absorbing material. Moreover, another layer such as a protective layer may be further provided as required.

Hereunder are exemplary aspects of the present invention.

<1> A near infrared ray absorbing material comprising at least a first compound having a spectral absorption maximum wavelength of 470 nm or less in a range of 270 to 1600 nm in solution, and a second compound represented by the following formula (II-1) or (II-2). By including the first compound having a spectral absorption maximum wavelength in addition to the second compound serving as a near infrared absorbent, the near infrared ray absorbing material has an excellent light resistance.

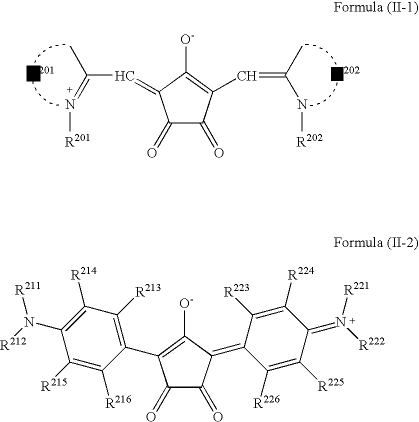

[In the formulas, R²⁰¹, R²⁰², R²¹¹, R²¹², R²²¹, and R²²² each represent, independently, a hydrogen atom, an aliphatic group, an aromatic group, or a heterocyclic group linked via a carbon atom. Z²⁰¹ and Z²⁰² represent a nonmetal atomic group necessary for forming a nitrogen-containing heterocycle. R²¹³ to R²¹⁶, and R²²³ to R²²⁶ represent a hydrogen atom or a substituent. R²¹³ and R²¹⁴, R²¹⁴ and R²¹¹, R²¹¹ and R²¹², R²¹² and R²¹⁵, R²¹⁵ and R²¹⁶, R²²³ and R²²⁴, R²²⁴ and R²²¹, R²²¹ and R²²², R²²² and R²²⁵, and R²²⁵ and R²²⁶ may be bonded with each other to form a ring.]

<2> A near infrared ray absorbing material according to <1>, wherein the spectral absorption maximum wavelength of the first compound is 430 nm or less.

<3> A near infrared ray absorbing material according to either <1> or <2>, wherein the spectral absorption maximum wavelength of the first compound is 410 nm or less.

<4> A near infrared ray absorbing material according to any one of <1> to <3>, wherein the spectral absorption maximum wavelength of the first compound is 380 nm or less.

Since the first compound is a compound having a spectral absorption maximum wavelength according to the above <2> to <4>, a near infrared ray absorbing material having a better light resistance can be produced.

<5> A near infrared ray absorbing material according to any one of <1> to <4>, wherein the first compound and the second compound are present together in the same layer. Since the first compound is present in the vicinity of the second compound, the light resistance can be further efficiently improved.

<6> A near infrared ray absorbing material according to any one of <1> to <5>,

wherein the first compound is represented by any one of the following formulas (I-1) to (I-5).

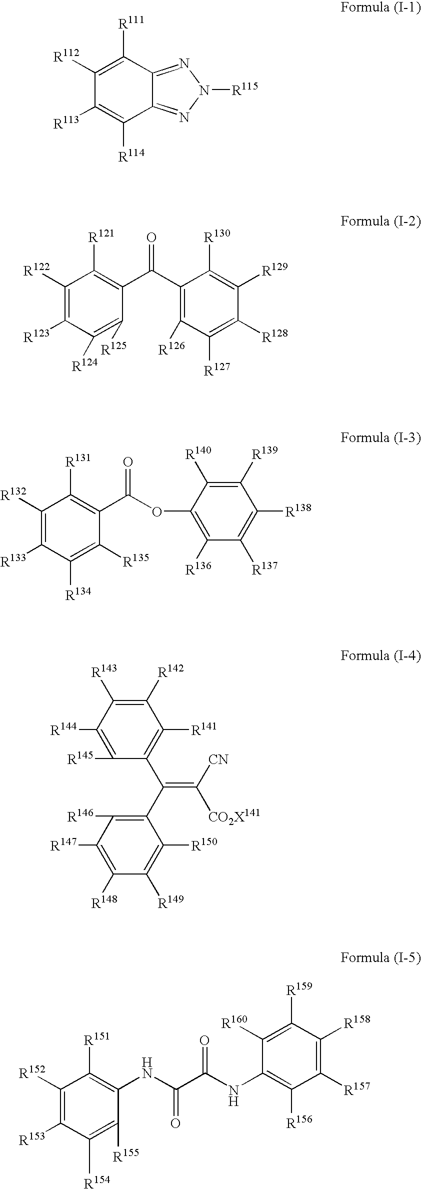

[In the formulas, R¹¹¹ to R¹¹⁴, R¹²¹ to R¹³⁰, R¹³¹ to R¹⁴⁰, R¹⁴¹ to R¹⁵⁰, and R¹⁵¹ to R¹⁶⁰ each represent, independently, a hydrogen atom or a substituent, R¹¹⁵ represents a hydrogen atom, an aliphatic group, an aromatic group, or a heterocyclic group linked via a carbon atom, and X¹⁴¹ represents a hydrogen atom, an aliphatic group, an aromatic group, or a heterocyclic group linked via a carbon atom.

Here, adjacent groups among R¹¹¹ to R¹¹⁴, R¹²¹ to R¹³⁰, R¹³¹ to R¹⁴⁰, R¹⁴¹ to R¹⁵⁰, and R¹⁵⁰ to R¹⁶⁰ substituted in a benzene ring in respective formulas, may be bonded with each other to form a ring.]

Since the first compound is the compound having a specific structure, the light resistance can be further efficiently improved.

<7> A near infrared ray absorbing material according to any one of <1> to <6>, comprising a plurality of the first compounds. By the interaction of the plurality of the first compounds, the light resistance can be further efficiently improved.

<8> A near infrared ray absorbing material according to <7>, wherein at least one type of the first compound is represented by the formula (I-1).

By including the first compound having an excellent ultraviolet ray absorbing power, the light resistance can be further efficiently improved.

<9> A near infrared ray absorbing material according to either <7> or <8>, wherein at least one type of the first compound is represented by any one of the formulas (I-2) to (I-5). By including the first compound having an excellent ultraviolet ray absorbing power, the light resistance can be further efficiently improved.

<10> A near infrared ray absorbing material according to any one of <1> to <9>, wherein the total number of moles of the first compound is 0.1 or more with respect to 1 mole of the second compound. By having the content ratio of the first compound with respect to the second compound within the above range, an excellent light resistance is shown.

<11> A near infrared ray absorbing filter, comprising a near infrared ray absorbing material according to any one of <1> to <10>. By using a near infrared ray absorbing material showing an excellent light resistance, a near infrared ray absorbing filter satisfying both of light resistance and near infrared ray absorbing power can be produced.

### EXAMPLES

Hereunder is a detailed description of the present invention, with reference to examples. However, the present invention is not limited to these examples.

For the sake of convenience, specific examples of the compound (I) are referred to as compounds (I-a) and (I-b), and specific examples of the compound (II) are referred to as compound (II-a). Moreover, the term “structure” in the following tables means the corresponding exemplary compound number.

### Example 1

### Production of Near Infrared Ray Absorbing Filter

To 10 g of polystyrene, the compounds (I-a) and (I-b) having the structure and the quantity shown in the following Table 1 and Table 2, and 0.1 g of the exemplary compound (II-22), was added 100 ml of chloroform. The mixture was stirred and dissolved at 40° C. for 15 minutes, and then coated on a glass plate, followed by drying with a fan at room temperature, to produce a sample

### (Light Resistance Test)

The sample obtained in example 1 was irradiated with 95,000 lux by a xenon lamp for 3 days. The absorbance at the spectral absorption maximum wavelength of the exemplary compound (II-22) was measured to obtain the remaining ratio with respect to the absorbance at the spectral absorption maximum wavelength before irradiation, and the light resistance (lightfastness) was evaluated. The results are shown in Table 1 and Table 2.

TABLE 1

| | Compound (I-a) | | Compound (I-b) | | | |
|---|---|---|---|---|---|---|
| Test | | (mole ratio | | (mole ratio | Light | |
| No. | Structure | to II-22) | Structure | to II-22) | resistance | Comment |
| 100 | Not added | | Not added | | 0.06 | Comparative Example |
| 101 | (I-9) | 0.05 | Not added | | 0.08 | Present Invention |
| 102 | (I-9) | 0.1 | Not added | | 0.24 | Present Invention |
| 103 | (I-9) | 0.2 | Not added | | 0.36 | Present Invention |
| 104 | (I-9) | 0.4 | Not added | | 0.40 | Present Invention |
| 105 | (I-9) | 1 | Not added | | 0.42 | Present Invention |
| 106 | Not added | | (I-14) | 0.05 | 0.07 | Present Invention |
| 107 | Not added | | (I-14) | 0.1 | 0.21 | Present Invention |
| 108 | Not added | | (I-14) | 0.2 | 0.33 | Present Invention |
| 109 | Not added | | (I-14) | 0.4 | 0.37 | Present Invention |
| 110 | Not added | | (I-14) | 1 | 0.40 | Present Invention |
| 111 | (I-9) | 0.025 | (I-14) | 0.025 | 0.09 | Present Invention |
| 112 | (I-9) | 0.05 | (I-14) | 0.05 | 0.38 | Present Invention |
| 113 | (I-9) | 0.1 | (I-14) | 0.1 | 0.46 | Present Invention |
| 114 | (I-9) | 0.2 | (I-14) | 0.2 | 0.56 | Present Invention |
| 115 | (I-9) | 0.5 | (I-14) | 0.5 | 0.60 | Present Invention |
| 116 | Not added | | (I-18) | 0.05 | 0.08 | Present Invention |
| 117 | Not added | | (I-18) | 0.1 | 0.23 | Present Invention |
| 118 | Not added | | (I-18) | 0.2 | 0.33 | Present Invention |
| 119 | Not added | | (I-18) | 0.4 | 0.37 | Present Invention |
| 120 | Not added | | (I-18) | 1 | 0.39 | Present Invention |
| 121 | (I-9) | 0.025 | (I-18) | 0.025 | 0.08 | Present Invention |
| 122 | (I-9) | 0.05 | (I-18) | 0.05 | 0.39 | Present Invention |
| 123 | (I-9) | 0.1 | (I-18) | 0.1 | 0.44 | Present Invention |
| 124 | (I-9) | 0.2 | (I-18) | 0.2 | 0.49 | Present Invention |
| 125 | (I-9) | 0.5 | (I-18) | 0.5 | 0.50 | Present Invention |
| 126 | Not added | | (I-41) | 0.05 | 0.08 | Present Invention |
| 127 | Not added | | (I-41) | 0.1 | 0.16 | Present Invention |
| 128 | Not added | | (I-41) | 0.2 | 0.25 | Present Invention |
| 129 | Not added | | (I-41) | 0.4 | 0.28 | Present Invention |
| 130 | Not added | | (I-41) | 1 | 0.31 | Present Invention |
| 131 | (I-9) | 0.025 | (I-41) | 0.025 | 0.09 | Present Invention |
| 132 | (I-9) | 0.05 | (I-41) | 0.05 | 0.48 | Present Invention |
| 133 | (I-9) | 0.1 | (I-41) | 0.1 | 0.59 | Present Invention |
| 134 | (I-9) | 0.2 | (I-41) | 0.2 | 0.66 | Present Invention |
| 135 | (I-9) | 0.5 | (I-41) | 0.5 | 0.68 | Present Invention |

TABLE 2

| | Compound (I-a) | | Compound (I-b) | | | |
|---|---|---|---|---|---|---|
| Test | | (mole ratio | | (mole ratio | Light | |
| No. | Structure | to II-22) | Structure | to II-22) | resistance | Comment |
| 136 | Not added | | (I-52) | 0.05 | 0.08 | Present Invention |
| 137 | Not added | | (I-52) | 0.1 | 0.17 | Present Invention |
| 138 | Not added | | (I-52) | 0.2 | 0.24 | Present Invention |
| 139 | Not added | | (I-52) | 0.4 | 0.28 | Present Invention |
| 140 | Not added | | (I-52) | 1 | 0.33 | Present Invention |
| 141 | (I-9) | 0.025 | (I-52) | 0.025 | 0.13 | Present Invention |
| 142 | (I-9) | 0.05 | (I-52) | 0.05 | 0.40 | Present Invention |
| 143 | (I-9) | 0.1 | (I-52) | 0.1 | 0.58 | Present Invention |
| 144 | (I-9) | 0.2 | (I-52) | 0.2 | 0.64 | Present Invention |
| 145 | (I-9) | 0.5 | (I-52) | 0.5 | 0.68 | Present Invention |
| 146 | Not added | | (I-71) | 0.05 | 0.07 | Present Invention |
| 147 | Not added | | (I-71) | 0.1 | 0.19 | Present Invention |
| 148 | Not added | | (I-71) | 0.2 | 0.21 | Present Invention |
| 149 | Not added | | (I-71) | 0.4 | 0.24 | Present Invention |
| 150 | Not added | | (I-71) | 1 | 0.28 | Present Invention |
| 151 | (I-9) | 0.025 | (I-71) | 0.025 | 0.11 | Present Invention |
| 152 | (I-9) | 0.05 | (I-71) | 0.05 | 0.39 | Present Invention |
| 153 | (I-9) | 0.1 | (I-71) | 0.1 | 0.55 | Present Invention |
| 154 | (I-9) | 0.2 | (I-71) | 0.2 | 0.62 | Present Invention |
| 155 | (I-9) | 0.5 | (I-71) | 0.5 | 0.65 | Present Invention |
| 156 | Not added | | (I-93) | 0.05 | 0.08 | Present Invention |
| 157 | Not added | | (I-93) | 0.1 | 0.14 | Present Invention |
| 158 | Not added | | (I-93) | 0.2 | 0.18 | Present Invention |
| 159 | Not added | | (I-93) | 0.4 | 0.25 | Present Invention |
| 160 | Not added | | (I-93) | 1 | 0.27 | Present Invention |
| 161 | (I-9) | 0.025 | (I-93) | 0.025 | 0.10 | Present Invention |
| 162 | (I-9) | 0.05 | (I-93) | 0.05 | 0.35 | Present Invention |
| 163 | (I-9) | 0.1 | (I-93) | 0.1 | 0.49 | Present Invention |
| 164 | (I-9) | 0.2 | (I-93) | 0.2 | 0.56 | Present Invention |
| 165 | (I-9) | 0.5 | (I-93) | 0.5 | 0.62 | Present Invention |

As shown in Table 1 and Table 2, the light resistance was improved by adding the compound (I-a) in both cases. Moreover, rather than the case where only one type of compound (I-a) was added, better light resistance was shown and the effect was very significant in the case where a plurality of compounds (I-a) having the same number of moles in total were added. Furthermore, all samples of the present invention after the test showed a weaker brown color than those of the comparative samples, by visual observation.

### Example 2

To 10 g of polystyrene, the compounds (I-a) and (I-b) having the structure and the quantity shown in the following Table 3 and Table 4, and 0.1 g of the compound (II-a), was added 100 ml of chloroform. The mixture was stirred and dissolved at 40° C. for 15 minutes, and then coated on a glass plate, followed by drying with a fan at room temperature, to produce a sample

### (Light Resistance Test)

The sample obtained in example 3 was irradiated with 95,000 lux by a xenon lamp for 3 days. The absorbance at the spectral absorption maximum wavelength of the compound (II-a) was measured to obtain the remaining ratio with respect to the absorbance at the spectral absorption maximum wavelength before irradiation, and the light resistance (lightfastness) was evaluated. The results are shown in Table 3 and Table 4.

TABLE 3

| | Compound (I-a) | | Compound (I-b) | | | | |
|---|---|---|---|---|---|---|---|
| Test | | (mole ratio | | (mole ratio | Compound (II-a) | Light | |
| No. | Structure | to II-22) | Structure | to II-22) | Structure | resistance | Comment |
| 200 | Not added | | Not added | | (II-4) | 0.05 | Comparative Example |
| 201 | (I-11) | 0.05 | Not added | | (II-4) | 0.06 | Present Invention |
| 202 | (I-11) | 0.1 | Not added | | (II-4) | 0.11 | Present Invention |
| 203 | (I-11) | 0.2 | Not added | | (II-4) | 0.25 | Present Invention |
| 204 | (I-11) | 0.4 | Not added | | (II-4) | 0.29 | Present Invention |
| 205 | (I-11) | 1 | Not added | | (II-4) | 0.32 | Present Invention |
| 206 | Not added | | (I-41) | 0.05 | (II-4) | 0.06 | Present Invention |
| 207 | Not added | | (I-41) | 0.1 | (II-4) | 0.10 | Present Invention |
| 208 | Not added | | (I-41) | 0.2 | (II-4) | 0.17 | Present Invention |
| 209 | Not added | | (I-41) | 0.4 | (II-4) | 0.20 | Present Invention |
| 210 | Not added | | (I-41) | 1 | (II-4) | 0.24 | Present Invention |
| 211 | (I-11) | 0.025 | (I-41) | 0.025 | (II-4) | 0.07 | Present Invention |
| 212 | (I-11) | 0.05 | (I-41) | 0.05 | (II-4) | 0.22 | Present Invention |
| 213 | (I-11) | 0.1 | (I-41) | 0.1 | (II-4) | 0.34 | Present Invention |
| 214 | (I-11) | 0.2 | (I-41) | 0.2 | (II-4) | 0.38 | Present Invention |
| 215 | (I-11) | 0.5 | (I-41) | 0.5 | (II-4) | 0.42 | Present Invention |
| 216 | (I-57) | 0.05 | Not added | | (II-4) | 0.06 | Present Invention |
| 217 | (I-57) | 0.1 | Not added | | (II-4) | 0.11 | Present Invention |
| 218 | (I-57) | 0.2 | Not added | | (II-4) | 0.17 | Present Invention |
| 219 | (I-57) | 0.4 | Not added | | (II-4) | 0.22 | Present Invention |
| 220 | (I-57) | 1 | Not added | | (II-4) | 0.30 | Present Invention |
| 221 | (I-57) | 0.025 | (I-41) | 0.025 | (II-4) | 0.08 | Present Invention |
| 222 | (I-57) | 0.05 | (I-41) | 0.05 | (II-4) | 0.26 | Present Invention |
| 223 | (I-57) | 0.1 | (I-41) | 0.1 | (II-4) | 0.31 | Present Invention |
| 224 | (I-57) | 0.2 | (I-41) | 0.2 | (II-4) | 0.36 | Present Invention |
| 225 | (I-57) | 0.5 | (I-41) | 0.5 | (II-4) | 0.40 | Present Invention |

TABLE 4

| | Compound (I-a) | | Compound (I-b) | | | | |
|---|---|---|---|---|---|---|---|
| Test | | (mole ratio | | (mole ratio | Compound (II-a) | Light | |
| No. | Structure | to II-22) | Structure | to II-22) | Structure | resistance | Comment |
| 226 | Not added | | Not added | | (II-18) | 0.06 | Comparative Example |
| 227 | (I-18) | 0.05 | Not added | | (II-18) | 0.08 | Present Invention |
| 228 | (I-18) | 0.1 | Not added | | (II-18) | 0.13 | Present Invention |
| 229 | (I-18) | 0.2 | Not added | | (II-18) | 0.20 | Present Invention |
| 230 | (I-18) | 0.4 | Not added | | (II-18) | 0.27 | Present Invention |
| 231 | (I-18) | 1 | Not added | | (II-18) | 0.31 | Present Invention |
| 232 | Not added | | (I-71) | 0.05 | (II-18) | 0.08 | Present Invention |
| 233 | Not added | | (I-71) | 0.1 | (II-18) | 0.14 | Present Invention |
| 234 | Not added | | (I-71) | 0.2 | (II-18) | 0.23 | Present Invention |
| 235 | Not added | | (I-71) | 0.4 | (II-18) | 0.32 | Present Invention |
| 236 | Not added | | (I-71) | 1 | (II-18) | 0.35 | Present Invention |
| 237 | (I-18) | 0.025 | (I-71) | 0.025 | (II-18) | 0.10 | Present Invention |
| 238 | (I-18) | 0.05 | (I-71) | 0.05 | (II-18) | 0.33 | Present Invention |
| 239 | (I-18) | 0.1 | (I-71) | 0.1 | (II-18) | 0.42 | Present Invention |
| 240 | (I-18) | 0.2 | (I-71) | 0.2 | (II-18) | 0.46 | Present Invention |
| 241 | (I-18) | 0.5 | (I-71) | 0.5 | (II-18) | 0.49 | Present Invention |
| 242 | Not added | | Not added | | (II-24) | 0.06 | Comparative Example |
| 243 | (I-47) | 0.05 | Not added | | (II-24) | 0.07 | Present Invention |
| 244 | (I-47) | 0.1 | Not added | | (II-24) | 0.09 | Present Invention |
| 245 | (I-47) | 0.2 | Not added | | (II-24) | 0.14 | Present Invention |
| 246 | (I-47) | 0.4 | Not added | | (II-24) | 0.24 | Present Invention |
| 247 | (I-47) | 1 | Not added | | (II-24) | 0.31 | Present Invention |
| 248 | Not added | | (I-17) | 0.05 | (II-24) | 0.08 | Present Invention |
| 249 | Not added | | (I-17) | 0.1 | (II-24) | 0.12 | Present Invention |
| 250 | Not added | | (I-17) | 0.2 | (II-24) | 0.18 | Present Invention |
| 251 | Not added | | (I-17) | 0.4 | (II-24) | 0.22 | Present Invention |
| 252 | Not added | | (I-17) | 1 | (II-24) | 0.28 | Present Invention |
| 253 | (I-47) | 0.025 | (I-17) | 0.025 | (II-24) | 0.10 | Present Invention |
| 254 | (I-47) | 0.05 | (I-17) | 0.05 | (II-24) | 0.25 | Present Invention |
| 255 | (I-47) | 0.1 | (I-17) | 0.1 | (II-24) | 0.33 | Present Invention |
| 256 | (I-47) | 0.2 | (I-17) | 0.2 | (II-24) | 0.38 | Present Invention |
| 257 | (I-47) | 0.5 | (I-17) | 0.5 | (II-24) | 0.41 | Present Invention |

As shown in Table 3 and Table 4, the light resistance was improved by adding the compound (I-a) with various different compounds (II-a), in both cases. Moreover, rather than the case where only one type of compound (I-a) was added, better light resistance was shown and the effect was very significant in the case where a plurality of compounds (I-a) having the same number of moles in total were added. Furthermore, all samples of the present invention after the test showed a weaker brown color than those of the comparative samples, by visual observation.

The physical properties of the compounds used in the examples of the present invention are shown below.

TABLE 5

| Exemplary compound | λmax (ε) | Measurement solvent |
|---|---|---|
| I-9 | 349 nm (1.52 × 10⁴) | ethyl acetate |
| I-11 | 348 nm (1.67 × 10⁴) | ethyl acetate |
| I-14 | 348 nm (1.66 × 10⁴) | ethyl acetate |
| I-17 | 338 nm (1.59 × 10⁴) | ethyl acetate |
| I-18 | 339 nm (1.62 × 10⁴) | ethyl acetate |
| I-41 | 351 nm (1.91 × 10⁴) | ethyl acetate |
| I-47 | 353 nm (2.01 × 10⁴) | ethyl acetate |
| I-52 | 308 nm (4.70 × 10³) | N,N-dimethylformamide |
| I-57 | 309 nm (4.30 × 10³) | N,N-dimethylformamide |
| I-71 | 299 nm (1.28 × 10⁴) | ethyl acetate |
| I-93 | 297 nm (1.45 × 10⁴) | ethyl acetate |
| II-4 | 806 nm | methanol |
| II-18 | 832 nm | N,N-dimethylformamide |
| II-22 | 847 nm | tetrahydrofuran |
| II-24 | 828 nm | N,N-dimethylformamide |

In naphthalocyanine dyes having a specific structure described in JP-A No. 2-4685, JP-A No. 2-43269, and JP-A No. 2-138382, it was difficult to satisfy both absorption wavelength and another physical property such as solubility.

Moreover, methods described in JP-A No. 11-167350, JP-A No. 2001-133624, and JP-A No. 2005-181966 are all related to a phthalocyanine compound, a diimonium compound, and the like, and the degree of suppression is insufficient. Thus techniques further improving the light resistance have been in demand. Furthermore, a valuable method for suppressing the decomposition of a compound having a croconium skeleton as a near infrared ray absorbing dye has not been reported yet.

According to the present invention, there can be provided a near infrared ray absorbing material in which light resistance, near infrared ray absorbing power, and less discoloration after light deterioration can be all satisfied, and a near infrared ray absorbing filter comprising the near infrared ray absorbing material.

What is claimed is:

1. A near infrared ray absorbing material comprising at least a first compound having a spectral absorption maximum wavelength of 470 nm or less in a range of 270 to 1600 nm in solution, and a second compound represented by the following formula (II-1) or (II-2): 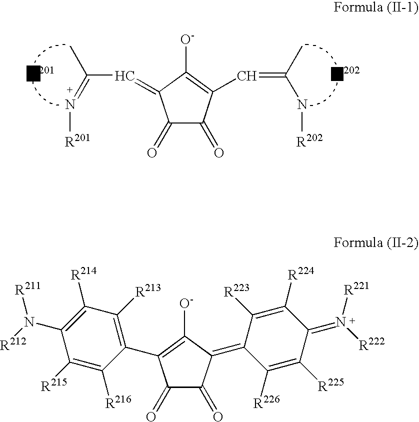
where in the formulas, R²⁰¹, R²⁰², R²¹¹, R²¹², R²²¹, and R²²² each represent, independently, a hydrogen atom, an aliphatic group, an aromatic group, or a heterocyclic group linked via a carbon atom; Z²⁰¹ and Z²⁰² represent a nonmetal atomic group necessary for forming a nitrogen-containing heterocycle; R²¹³ to R²¹⁶, and R²²³ to R²²⁶ represent a hydrogen atom or a substituent; and R²¹³ and R²¹⁴, R²¹⁴ and R²¹¹, R²¹¹ and R²¹², R²¹² and R²¹⁵, R²¹⁵ and R²¹⁶, R²²³ and R²²⁴, R²²⁴ and R²²¹, R²²¹ and R²²², R²²² and R²²⁵, and R²²⁵ and R²²⁶ may be bonded with each other to form a ring.

2. A near infrared ray absorbing material according to claim 1, wherein the spectral absorption maximum wavelength of the first compound is 430 nm or less.

3. A near infrared ray absorbing material according to claim 1, wherein the spectral absorption maximum wavelength of the first compound is 410 nm or less.

4. A near infrared ray absorbing material according to claim 1, wherein the spectral absorption maximum wavelength of the first compound is 380 nm or less.

5. A near infrared ray absorbing material according to claim 1, wherein the first compound and the second compound are present together in a same layer.

6. A near infrared ray absorbing material according to claim 1, wherein the first compound is represented by any one of the following formulas (I-1) to (I-5): 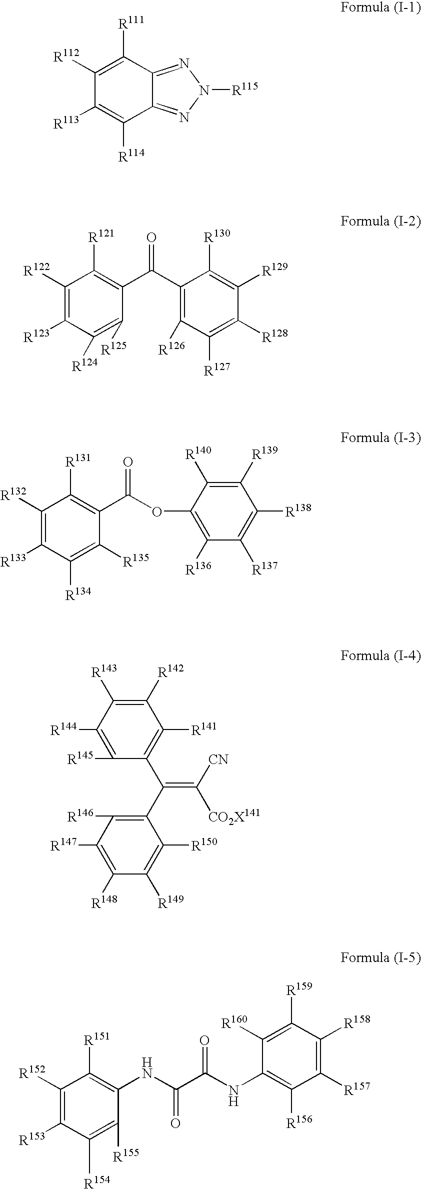
where in the formulas, R¹¹¹ to R¹¹⁴, R¹²¹ to R¹³⁰, R¹³¹ to R¹⁴⁰, R¹⁴¹ to R¹⁵⁰, and R¹⁵¹ to R¹⁶⁰ each represent, independently, a hydrogen atom or a substituent, R¹¹⁵ represents a hydrogen atom, an aliphatic group, an aromatic group, or a heterocyclic group linked via a carbon atom, and X¹⁴¹ represents a hydrogen atom, an aliphatic group, an aromatic group, or a heterocyclic group linked via a carbon atom; and adjacent groups among R¹¹¹ to R¹¹⁴, R¹²¹ to R¹³⁰, R¹³¹ to R¹⁴⁰, R¹⁴¹ to R¹⁵⁰, and R¹⁵¹ to R¹⁶⁰ substituted in a benzene ring in respective formulas, may be bonded with each other to form a ring.

7. A near infrared ray absorbing material according to claim 1, comprising a plurality of the first compounds.

8. A near infrared ray absorbing material according to claim 7, wherein at least one of the first compound is represented by the formula (I-1).

9. A near infrared ray absorbing material according to claim 7, wherein at least one of the first compound is represented by any one of the formulas (I-2) to (I-5).

10. A near infrared ray absorbing material according to claim 1, wherein the total number of moles of the first compound is 0.1 or more with respect to 1 mole of the second compound.

11. A near infrared ray absorbing filter, comprising a near infrared ray absorbing material according to claim 1.
